(19) United States
(12) Patent Application Publication
(10) Pub. No.: US 2013/0315940 A1
(43) Pub. Date: Nov. 28, 2013

(54) TREATMENT OF LIVER CANCER

(71) Applicants: Rajiv Jalan, Chislehurst (GB); Fatma El-zahraa Ammar Saleh Mohamed, London (GB)

(72) Inventors: Rajiv Jalan, Chislehurst (GB); Fatma El-zahraa Ammar Saleh Mohamed, London (GB)

(73) Assignee: Rajiv Jalan, Chislehurst (GB)

(21) Appl. No.: 13/818,018

(22) PCT Filed: Aug. 19, 2011
(86) PCT No.: PCT/GB2011/001254

§ 371 Date: Aug. 5, 2013

(30) Foreign Application Priority Data
Aug. 20, 2010 (GB) 1014026.7

(51) Int. Cl. G01N 33/574

(52) U.S. Cl. CPC G01N 33/57438
USPC 424/184.1; 514/313; 514/44 R; 435/7.23

(57) ABSTRACT

The present invention derives from the finding that increased levels of Toll related receptor 9 (TLR9) and Toll related receptor 7 (TLR7) are associated with liver cancer and in particular hepatocellular carcinoma. Accordingly, the invention provides TLR9 and TLR7 antagonists for use in a method of treating liver cancer. The invention provides other strategies such as modulation of endosomal signalling by using compounds such as chloroquine to prevent or treat such liver cancer. The invention also provides agents capable of inducing an immune response against TLR9 and TLR7 for use in a method of treating liver cancer. The presence of TLR9 and TLR7 expression may be indicative of the presence of liver cancer and the amount of TLR9 and TLR7 expression may be indicative of the rate of growth or spreading of the cancer.

### FIELD OF THE INVENTION

The present invention derives from the unexpected finding that expression of Toll like receptor 9 (TLR9) and Toll like receptor 7 (TLR7) is increased in liver cancer. The level of expression of TLR9 correlates with the proliferation of the cancer cells. The present invention utilises these findings to identify and provide TLR9 and TLR7 antagonists that may be used in the treatment, prevention or diagnosis of liver cancer, for example in the treatment of hepatocellular carcinoma or cholangiocarcinoma.

### BACKGROUND TO THE INVENTION

Hepatocellular carcinoma (HCC) is one of the most common malignancies worldwide, being the fifth most common cancer type and the third most common cause of cancer death. There are around one million new cases of HCC annually, with nearly half a million deaths caused by HCC. The prognosis of HCC is poor and the survival rate is only about 3-5%.

About 70-90% of HCC cases are found in individuals having previous cirrhosis and inflammation. Risk factors for HCC include viral hepatitis caused by infection with hepatitis B or hepatitis C, heavy alcohol consumption, obesity, non-alcoholic steatohepatitis, exposure to carcinogenic toxins (e.g. aflatoxins) or carcinogenic drugs, hemochromatosis, autoimmune hepatitis, α1-antitrypsin deficiency and glycogen storage disease type 1.

Unlike other solid tumors, the specific sequence of events that mediate hepatocarcinogenesis is not known. HCC usually progresses from chronic hepatitis, to cirrhosis, to dysplastic nodules (low and high grade) and then to malignant tumors.

### SUMMARY OF THE INVENTION

It has surprisingly been found that expression of TLR9 and TLR7 is seen in liver cancer, particularly hepatocellular carcinoma (HCC). Expression of TLR9 and TLR7 is seen in the cytoplasm, plasma membrane, endosome and nucleus of such tissue. Expression of TLR7 and TLR9 is seen in the cytoplasm in HCC, but not in other liver diseases such as cirrhosis. Furthermore, it has been found that the level of TLR9 expression in such cancer tissue correlates with the level of proliferation seen in that tissue. Expression of TLR9 is particularly high at the proliferative edges of an expanding liver tumor.

The present invention therefore proposes therapies for liver cancer based on this finding. Accordingly, the invention provides an antagonist of Toll like receptor 9 (TLR9) and/or Toll like receptor 7 (TLR7) for use in a method of treating or preventing liver cancer. Also provided is an agent capable of stimulating an immune response against TLR9 and/or TLR7 for use in a method of treatment or prevention of liver cancer.

Similarly, the invention provides the use of an antagonist of TLR9 and/or TLR7 or an agent capable of stimulating an immune response against TLR9 and/or TLR7 in the manufacture of a medicament for use in the treatment or prevention of liver cancer.

The invention also provides a method of treating or preventing liver cancer in an individual in need thereof, said method comprising a step of administering to said individual (a) an antagonist of TLR9, and/or (b) an antagonist of Toll like receptor 7 (TLR7), and/or (c) an antagonist of both TLR9 and TLR7, and/or (d) an agent capable of stimulating an immune response against TLR9 in the individual and/or (e) an agent capable of stimulating an immune response against TLR7 in the individual. The antagonist may lead to: (a) decreased expression of TLR9 in the liver of the individual; and/or (b) decreased levels of TLR9 in the liver of the individual; and/or (c) decreased activity of TLR9 in the liver of the individual. The antagonist may additionally or alternatively lead to: (a) decreased expression of TLR7 in the liver of the individual; and/or (b) decreased levels of TLR7 in the liver of the individual; and/or (c) decreased activity of TLR7 in the liver of the individual. The antagonist may be selected from DV1079, IMO3100, CPG52364 and IRS954. The antagonist may be a modulator of endosomal signalling such as chloroquine or hydroxychloroquine or quinacrine or bafilomycin A.

The liver cancer may be hepatocellular carcinoma (HCC) or cholangiocarcinoma.

The invention also provides a method of detecting liver cancer comprising (a) providing a sample of liver from an individual, and (b) determining whether TLR9 and/or TLR7 is expressed in said sample, wherein the expression of TLR9 and/or TLR7 in said sample indicates that the individual may be suffering from liver cancer. Also provided is a method of determining whether a liver cancer is spreading comprising (a) providing a sample from the edge of a liver cancer in the individual, and (b) determining the level of TLR9 expressed in the cancer, wherein a greater level of TLR9 expressed at the edge of the cancer indicates that the cancer is more likely to be spreading. Similarly, the invention provides a method of determining the rate of growth of a liver cancer comprising (a) providing a sample of liver from an individual including some tumor tissue, and (b) assessing the level of TLR9 expression in said sample, wherein the level of TLR9 in said sample is indicative of the rate of growth of the cancer. Preferably in such methods said liver cancer is hepatocellular carcinoma or cholangiocarcinoma.

The invention also provides a method of identifying an agent suitable for use in treating or preventing liver cancer, the method comprising determining whether a test agent is capable of decreasing the amount or activity of TLR9 and/or TLR7, wherein the ability to decrease the amount or activity of TLR9 and/or TLR7 indicates that the compound may be suitable for use in treating liver cancer.

### BRIEF DESCRIPTION OF THE DRAWINGS

FIG. 1: Staining for TLR9 in liver tissue sections. A: TLR9 shows a difference in staining intensity between tumour part and cirrhotic part. B: Membranous staining of the hepatic cells in cirrhosis. C: Negative staining for TLR9 in the non tumoural liver.

FIG. 2: Staining for TLR9 in liver tissue sections. A: TLR9 positive cytoplasmic staining graded +3. B: Corresponding Ki-67 positive staining in the same area showing high intensity of TLR9.

FIG. 3: A very close correlation exists between cytoplasmic staining for TLR9 and Ki-67 positivity.

FIG. 4A: TLR9 cytoplasmic staining and caspase 3 staining. FIG. 4B: TLR9 cytoplasmic staining and caspase 3 staining Good correlation exists between TLR9 staining and caspase 3 staining.

FIG. 5A: TLR9 expression in Huh7 cells after 24 h. FIG. 5B TLR9 expression in Huh7 cells after 48 h. FIG. 5C: TLR9 isotype in Huh7 cells. These indicate there the Hepatocellular carcinoma cell line HuH 7 contains TLR9.

FIG. 6A: TLR2 in Huh7 after 24 h. FIG. 6B TLR2 in Huh7 after 48 h. FIG. 6C: TLR2 isotype in Huh7. The expression of TLR2 is limited in Huh7 cells.

FIG. 7: Expression of TLR9 in hepatocellular carcinoma (HCC) compared with cirrhosis. The dark areas of staining represent positive staining for TLR9. TLR9 staining was markedly increased in the cancer which is not seen in the cirrhotic tissue.

FIG. 8 shows the significant correlation between the intensity of cytoplasmic TLR9 and the proliferation index which is measured by the positive ki-67 hepatocytes among 1000 hepatocytes; r=0.8, p<0.001.

FIG. 9 shows that TLR7 immunostaining is markedly increased in HCC (A), but is not present in cirrhosis (B).

FIG. 10 shows the effects of treatment with DEN and NMOR (which induce hepatocellular carcinoma). Under the microscope (A) and looking at macroscopic appearance of the liver (B), hepatocellular carcinoma was present after treatment with DEN and NMOR. C and D: no cancer was apparent either microscopically or macroscopically in the DEN, NMOR group that was treated with chloroquine.

FIG. 11A shows that tumor size was significantly less in the group treated with chloroquine (A). FIG. 11B: nodules in animals treated with chloroquine or control. The size of the nodules were significantly smaller in the group treated with chloroquine compared with controls.

FIG. 12 shows the effects on tumor size of treatment with the TLR7 and 9 inhibitor IRS 954 (Dynavax). The photographs on the left show nodules from control animals, the photographs on the right show nodules from inhibitor treated controls. The size of the tumours was significantly less in the IRS 954 treated group.

FIG. 13 shows that in the HCC cell line HuH7, TLR9 stimulator cpg was associated with evidence of proliferation which was significantly inhibited in the presence of iODN (TLR9 inhibitor) or chloroquine. B: TLR9 stimulation with cpg increased proliferation of HepG2 cells. C: treatment of HepG2 cells with chloroquine reduced cell proliferation.

FIG. 14 provides confocal images of TLR9 localisation in HuH7 cells. A: shows control localisation in the cytoplasm, plasma membrane, endosome and nucleus. B: treatment with the TLR9 stimulatory cpg shows increased TLR9 in the cytoplasm, plasma membrane, endosome and nucleus. C: treatment with the TLR9 inhibitory oliginucleotide (iODN) shows markedly reduced TLR9 expression in the cytoplasm, plasma membrane, endosome and nucleus.

FIG. 15 shows TLR7 localisation within the cell localised in the cytoplasm, plasma membrane, endosome and nucleus.

### DETAILED DESCRIPTION OF THE INVENTION

The Toll like receptors (TLRs) play an important role in innate immune responses, particularly against pathogen-associated molecular patterns. The TLRs are transmembrane receptors characterised by the presence of a leucine rich repeat in their extracellular domain and a toll/interleukin-1 receptor (TIR) domain in their intracellular domain.

Toll like receptor 9 (TLR9) is believed to recognise unmethylated CpG DNA motifs from the DNA bacteria and also unmethylated CpG in the viral genome. TLR9 is located on the endosome lysosome membrane. TLR9 is preferentially expressed in immune cell rich tissues, such as spleen, lymph node, bone marrow and peripheral blood leukocytes. Toll like receptor 7 (TLR7) recognises single stranded RNA in endosomes, which is a common feature of viral genomes which are internalised by macrophages. TLR7 is also located in the endosome. TLR7 is predominantly expressed in the lung, placenta and spleen.

The present invention lies in the finding that expression of TLR9 and TLR7 is seen in liver cancer tissue. Targeting of TLR9 and/or TLR7, such as by supplying TLR9 and/or TLR7 antagonists or by stimulating an endogenous immune response against TLR9 and/or TLR7 may therefore have particular utility in the treatment or prevention of liver cancer. Detection of TLR9 and/or TLR7 expression may also have utility in the diagnosis or prognosis of liver cancer.

### TLR9 and TLR7 Antagonists

The present invention relates to the antagonism of Toll-like receptor 9 (TLR9) and/or Toll like receptor 7 (TLR7). An antagonist of TLR9 and/or TLR7 may be any compound or molecule that inhibits or decreases the activity, function or amount of TLR9, TLR7 or both TLR9 and TLR7. Preferably the antagonist functions in cells, tissues or organs that express TLR9 and/or TLR7 such as in liver cancer cells. The antagonist may act preferentially in the liver, the liver tumor or may act at a number of locations including the liver and/or liver tumor. The antagonist may act preferentially in particular cell types such as inflammatory cells, platelets or neurons. Preferably the antagonist leads to a decrease in TLR9 and/or TLR7 activity, function or amount in the cells, tissues or organs of an individual to whom the antagonist is administered, such as in the liver or in tumor tissue within the liver of the individual. The antagonist may be targeted to the liver or to the liver tumor during administration as discussed further below.

Preferred antagonists are those that decrease the activity or amount of TLR9 and/or TLR7 by at least 10%, at least 20%, at least 30%, at least 40%, at least 50%, at least 60%, at least 70%, at least 80% or at least 90% compared to the amount seen in the absence of the antagonist. For example, decreases of these sizes may be seen in the liver, liver tumor tissue or liver tissue of a subject to whom the agonist has been administered. Decreases of these sizes may be seen in other tissues or organs of the individual where TLR9 and/or TLR7 is expressed.

An antagonist of TLR9 and/or TLR7 may reduce the activity or amount of TLR9 and/or TLR7 in liver tumor tissue to an amount or activity that is the same, similar to, or equivalent to, that seen in normal liver tissue. For example, as explained herein, the expression of TLR9 and TLR7 was found to be increased in association with hepatocellular carcinoma. Use of a TLR9 and/or TLR7 antagonist in accordance with the present invention may lead to a reduction in TLR9 and/or TLR7 expression in the liver tumor tissue, such as the hepatocellular carcinoma tissue of the individual being treated to a normal level, such as a level that would be seen or would be expected in an individual not suffering from liver cancer or hepatocellular carcinoma.

The antagonist may act specifically to antagonise TLR9 and/or TLR7. That is the antagonist may act specifically on TLR9, TLR7 or TLR7 and TLR9. The effect of the antagonist on TLR9 and/or TLR7 may be greater than any other biological effect of the antagonist. Such an antagonist may be specific to the inhibition of TLR9 and/or TLR7 that is it may decrease the activity of TLR9 and/or TLR7, but not other Toll like receptors. Such an antagonist may additionally or alternatively be specific to the expression of TLR9 and/or TLR7 that is it may decrease the expression of TLR9 and/or TLR7 but not other Toll like receptors. A specific antagonist for use in accordance with the present invention may be an antagonist of TLR9 and/or TLR7 as described herein that does not act as an antagonist of other Toll like receptor types. A specific antagonist for use in accordance with the present invention may act on TLR9 and/or TLR7 in preference to other Toll like receptor types. For example, an antagonist of TLR9 and/or TLR7 for use in accordance with the present invention may have one or more of the characteristics of an TLR9 and/or TLR7 antagonist as described herein, but may not have such characteristics in relation to other Toll like receptor types, or may have such characteristics to a lower level in relation to other Toll like receptor types when compared to TLR9 and/or TLR7. For example, an antagonist that decreases the activity of TLR9 and/or TLR7 may not decrease the activity of other Toll like receptor types, or may decrease the activity of other Toll like receptor types to a lesser extent, such as a lower percentage decrease, than its effect on TLR9 and/or TLR7. An antagonist that decreases the expression or amount of TLR9 and/or TLR7 may not decrease the expression or amount of other Toll like receptor types, or may decrease the expression of other Toll like receptor types to a lesser extent, such as a lower percentage decrease, than its effect on TLR9 and/or TLR7. A lower percentage decrease here may be, for example, a decrease of less than 25%, less than 15%, less than 10%, less than 5%, less than 2%, less than 1% or less than 0.1% when compared with the decrease in expression, amount or activity of TLR9 and/or TLR7.

By other Toll like receptor types herein is meant any receptor that is not TLR9 and/or TLR7. For example, the other Toll like receptor type may be one or more of TLR2 and TLR4, or any other TLR. Where the antagonist is an antagonist of TLR9, the other Toll like receptor may be TLR7. Where the antagonist is an antagonist of TLR7, the other Toll like receptor may be TLR9. Where the antagonist is an antagonist of TLR9 and TLR7, it may have the effects described herein for both TLR9 and TLR7, but may be specific for those two Toll like receptors, i.e. the other Toll like receptor as described above may be another Toll like receptor such as TLR4 or TLR2.

The other TLR may be any one of these other Toll like receptor types. The TLR9 and/or TLR7 antagonist may be specific to TLR9 and/or TLR7 as discussed above in comparison to its effects on any other Toll like receptor type. For example, the TLR9 and/or TLR7 antagonist may be specific to TLR9 and/or TLR7 in comparison to TLR2 or TLR4.

The other Toll like receptor may be more than one of these Toll like receptor types. For example, the effects of the antagonist on a TLR9 and/or TLR7 receptor may be specific, as discussed above, when compared to the effects of that agent on TLR2, when compared to the effects of that agent on TLR4, or when compared to the effects of that agent when compared to other Toll like receptors that are not TLR9 and/or TLR7. The effects of the antagonist on a TLR9 and/or TLR7 receptor may be specific as discussed above when compared to all other classes of TLR that are present.

The specificity of the TLR9 and/or TLR7 antagonist may apply within the whole body of the individual to be treated, that is the actions of the TLR9 and/or TLR7 antagonist may be specific as discussed above throughout the body of the individual. The specificity of the TLR9 and/or TLR7 antagonist may apply within particular tissues of the individual, such as the liver, kidneys or heart. That is, in one embodiment, the TLR9 antagonist may act specifically to antagonise TLR9 and/or TLR7 as discussed above within the liver or within tumor tissue in the liver of the individual being treated.

The TLR9 and/or TLR7 antagonist may therefore be a specific antagonist of TLR9 and/or TLR7 as described above. For example, the TLR9 and/or TLR7 antagonist may not be an antagonist of TLR2, or may have no significant effect on the activity or expression of TLR2. The TLR9 and/or TLR7 antagonist may not be an antagonist of TLR4 or may not have any significant effect on the activity or expression of TLR4. The TLR9 and/or TLR7 antagonist may not be an antagonist of any other TLR that is not TLR9 and/or TLR7 or may not have any significant effect on the activity or expression of any such other TLR.

Any agent capable of inhibiting the activity or function of TLR9 and/or TLR7 may be suitable for use in the methods of the present invention. Antagonists for use in accordance with the present invention may be direct or indirect antagonists of TLR9 and/or TLR7.

Direct antagonists are agents whose activity is directly on TLR9 and/or TLR7. For example, direct antagonists may be agents that act directly on the TLR9 and/or TLR7 receptor to decrease its activity. A direct antagonist may be an agent that disrupts TLR9 and/or TLR7 function or that destabilises the TLR9 and/or TLR7 receptor. A direct antagonist may decrease the amount of TLR9 and/or TLR7 by destroying or disrupting TLR9 and/or TLR7 molecules within the patient. A direct antagonist may be an agent that acts on the TLR9 and/or TLR7 gene, promoter or other gene regulatory regions to decrease expression of the TLR9 and/or TLR7. A direct antagonist may decrease expression of TLR9 and/or TLR7 by preventing or reducing expression from the endogenous TLR9 and/or TLR7 gene.

Any agent or molecule having the properties described above may be used as a TLR9 antagonist in accordance with the present invention. Examples of TLR9 and/or TLR7 antagonists or inhibitors that may be used in accordance with the present invention include the following:

Chloroquine, hydroxychloroquine, quinacrine and bafilomycin A inhibit TLR7 and TLR9 by modulating endosomal pH.

DV1079 is a bifunctional inhibitor of TLR7 and TLR9 being developed by GlaxoSmithKline for the treatment of immuno-inflammatory diseases such as lupus, psoriasis and rheumatoid arthritis.

IMO3100 is a compound that suppresses immune responses mediated through TLR7 and TLR9 and is being developed by Idera Pharmaceuticals Inc.

Oligodeoxynucleotide compounds containing unmethylated CpG dinucleotides that can act as antagonists of TLR9 are described in Yu et al (J. Med Chem, 2009, 52: 5108-5114), the contents of which are hereby incorporated by reference. Any such molecule may be used as a TLR9 antagonist in accordance with the present invention. For example, the inhibitory oligodeoxynucleotide (ttaggg)₄ was used in the Examples.

CPG52364 is a small molecule antagonist designed to inhibit TLRs 7, 8 and 9 (see WO 2008/152471, the contents of which are hereby incorporated by reference).

IRS954 is an oligonucleotide which is an IRS inhibitor and which specifically inhibits TLR7 and TLR9 induced inflammatory responses. This molecule is described in Barrat et al (Eur J Immunol 2007 37: 3582-3586) and in Barrat et al J Exp Med 2005 202: 1131-1139), the contents of which are hereby incorporated by reference.

The TLR9 and/or TLR7 antagonist may be a molecule that is capable of binding to, and preventing or disrupting the activity of TLR9 and/or TLR7.

Accordingly, one group of TLR9 and/or TLR7 antagonists for use in accordance with this invention are anti-TLR9 and/or anti-TLR7 antibodies. Such an antibody may be monoclonal or polyclonal or may be an antigen-binding fragment thereof. For example, an antigen-binding fragment may be or comprise a F(ab)2, Fab or Fv fragment, i.e. a fragment of the “variable” region of the antibody, which comprises the antigen binding site. An antibody or fragment thereof may be a single chain antibody, a chimeric antibody, a CDR grafted antibody or a humanised antibody.

An antibody may be directed to the TLR9 and/or TLR7 molecule, i.e. it may bind to epitopes present on TLR9 and/or TLR7 and thus bind selectively and/or specifically to TLR9 and/or TLR7. An antibody may be directed to another molecule that is involved in the expression and/or activity of TLR9 and/or TLR7. For example, a polyclonal antibody may be produced which has a broad spectrum effect against one or more epitopes on TLR9 and/or TLR7 and/or one or more other molecules that are involved in the expression and/or activity of TLR9 and/or TLR7.

Antibodies can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

An antibody, or other compound, “specifically binds” to a molecule when it binds with preferential or high affinity to the molecule for which it is specific but does substantially bind not bind or binds with only low affinity to other molecules. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

The TLR9 and/or TLR7 antagonist may be an antisense oligonucleotide, such as an antisense oligonucleotide against the gene encoding an TLR9 and/or TLR7 protein.

The term “antisense oligonucleotide” as used herein means a nucleotide sequence that is complementary to the mRNA for a desired gene. Such an antisense oligonucleotide may selectively hybridise with the desired gene. In the context of the present invention, the desired gene may be the gene encoding TLR9 and/or TLR7.

The TLR9 and/or TLR7 antagonist may modulate expression of the TLR9 and/or TLR7 gene. For example, the TLR9 and/or TLR7 antagonist may be a short interfering nucleic acid (siRNA) molecule, double stranded RNA (dsRNA), micro RNA, deoxyribose nucleic acid interference (DNAi) or short hairpin RNA (shRNA) molecule.

The TLR9 and/or TLR7 antagonist may be an inhibitory oligodeoxynucleotide molecule. For example, the oligodeoxynucleotide (ttaggg)₄ is used in the Examples to inhibit TLR9.

The term “selectively hybridise” as used herein refers to the ability of a nucleic acid to bind detectably and specifically to a second nucleic acid. Oligonucleotides selectively hybridise to target nucleic acid strands under hybridisation and wash conditions that minimise appreciable amounts of detectable binding to non-specific nucleic acids. High stringency conditions can be used to achieve selective hybridisation conditions as known in the art. Typically, hybridisation and washing conditions are performed at high stringency according to conventional hybridisation procedures. Washing conditions are typically 1-3×SSC, 0.1-1% SDS, 50-70° C. with a change of wash solution after about 5-30 minutes.

The TLR9 and/or TLR7 antagonist may be a nucleic acid molecule such as an antisense molecule or an aptamer. The nucleic acid molecule may bind a specific target molecule.

Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

The terms “nucleic acid molecule” and “polynucleotide” are used interchangeably herein and refer to a polymeric form of nucleotides of any length, either deoxyribonucleotides or ribonucleotides, or analogs thereof. A nucleic acid may comprise conventional bases, sugar residues and inter-nucleotide linkages, but may also comprise modified bases, modified sugar residues or modified linkages. A nucleic acid molecule may be single stranded or double stranded.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Aptamers may be generated using routine methods such as the Systematic Evolution of Ligands by EXonential enrichment (SELEX) procedure. SELEX is a method for the in vitro evolution of nucleic acid molecules with highly specific binding to target molecules. It is described in, for example, U.S. Pat. No. 5,654,151, U.S. Pat. No. 5,503,978, U.S. Pat. No. 5,567,588 and WO 96/38579. The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

The antagonist may target the TLR9 and/or TLR7 in a particular location within the cell. For example, the antagonist may target TLR9 and/or TLR7 when present in the cytoplasm or the endosome. For example, the antagonist may be a molecule that alters the activity of TLR9 and/or TLR7 within the endosome. The antagonist may alter signalling by TLR9 and/or TLR7 within the endosome. Such an antagonist may alter activity or signalling by TLR9 and/or TLR7 by altering conditions within the endosome, such as by altering the pH within the endosome. A group of such antagonists that may be used in accordance with the present invention are chloroquine, hydroxychloroquine, quinacrine and bafilomycin A.

Any of the antagonists described herein may therefore be used to antagonise TLR9 and/or TLR7, i.e. to decrease the amount of TLR9 and/or TLR7 that is present, and/or the activity or the function of the TLR9 and/or TLR7. This antagonism may take place in any location or tissue where TLR9 and/or TLR7 is present. The antagonism may take place in one or more organs selected from the brain, kidney, liver and heart. The antagonism may take place on cells expressing TLR9 and/or TLR7, such as inflammatory cells, platelets and/or neurons. Preferably these antagonising effects take place in the liver. Most preferably these antagonising effects take place in the tumor tissue within the liver.

An antagonist of TLR9 and/or TLR7 may be an agent that decreases the production of endogenous TLR9 and/or TLR7. For example, the agent may act within the cells of the subject to inhibit or prevent the expression of TLR9 and/or TLR7. Such an agent may be a transcription factor or enhancer that acts on the TLR9 and/or TLR7 gene to inhibit or prevent gene expression.

TLR9 and/or TLR7 Antagonists for Vaccination

The TLR9 and/or TLR7 antagonist may be an indirect antagonist. Such an indirect antagonist may be, for example, a molecule that is capable of inducing an anti-TLR9 and/or TLR7 response in the individual to be treated, such as an anti-TLR9 and/or TLR7 immune response.

For example, a TLR9 and/or TLR7 antagonist may be, or may comprise a TLR9 and/or TLR7 polypeptide such as a naturally occurring TLR9 and/or TLR7 polypeptide or a variant or fragment thereof that is capable of stimulating an immune response in vivo against endogenous TLR9 and/or TLR7, such as TLR9 and/or TLR7 expressed in a liver cancer. Such a TLR9 and/or TLR7 polypeptide should be capable of acting as an antigen and should include at least one functional epitope from the original TLR9 and/or TLR7 polypeptide.

An “antigen” refers to any agent, generally a macromolecule, which can elicit an immunological response in an individual. As used herein, “antigen” is generally used to refer to a polypeptide molecule or portion thereof which contains one or more epitopes. Furthermore, for the purposes of the present invention, an “antigen” includes a polypeptide having modifications, such as deletions, additions and substitutions (generally conservative in nature) to the native sequence, so long as the polypeptide maintains sufficient immunogenicity. These modifications may be deliberate, for example through site-directed mutagenesis, or may be accidental, such as through mutations of hosts which produce the antigens.

An “immune response” against an antigen of interest is the development in an individual of a humoral and/or a cellular immune response to that antigen. For purposes of the present invention, a “humoral immune response” refers to an immune response mediated by antibody molecules, while a “cellular immune response” is one mediated by T-lymphocytes and/or other white blood cells.

As used herein, the term “epitope” generally refers to the site on a target antigen which is recognised by an immune receptor such as a T-cell receptor and/or an antibody. Preferably it is a short peptide derived from or as part of a protein. However the term is also intended to include peptides with glycopeptides and carbohydrate epitopes. A single antigenic molecule, such as one of the four proteins described herein, may comprise several different epitopes. The term “epitope” also includes modified sequences of amino acids or carbohydrates which stimulate responses which recognise the whole organism.

It is advantageous if the selected epitope is specific to TLR9 and/or TLR7. For example, as discussed further above, the TLR9 and/or TLR7 antagonist may be specific to TLR9 and/or TLR7 in preference to one or more other TLRs. Epitopes can be identified from knowledge of the amino acid and corresponding DNA sequences of the peptide or polypeptide, as well as from the nature of particular amino acids (e.g., size, charge, etc.) and the codon dictionary, without undue experimentation. See, e.g., Ivan Roitt, Essential Immunology, 1988; Janis Kuby, Immunology, 1992 e.g., pp. 79-81. Some guidelines in determining whether a protein or an epitope of interest will stimulate a response, include: peptide length—the peptide should be at least 8 or 9 amino acids long to fit into the MHC class I complex and at least 8-25, such at least as 13-25 amino acids long to fit into a class II MHC complex. These lengths are the minimum for the peptide to bind to the respective MHC complex. It is preferred for the peptides to be longer than these lengths because cells may cut peptides. The peptide should contain an appropriate anchor motif which will enable it to bind to the various class I or class II molecules with high enough specificity to generate an immune response. This can be done, without undue experimentation, by comparing the sequence of the protein of interest with published structures of peptides associated with the MHC molecules. Thus, the skilled artisan can ascertain an epitope of interest by comparing the protein sequence with sequences listed in the protein database.

Polypeptide “fragments” for use as TLR9 or TLR7 antagonists may be made by truncation, e.g. by removal of one or more amino acids from the N and/or C-terminal ends of a TLR9 or TLR7 polypeptide. Up to 10, up to 20, up to 30, up to 40 or more amino acids may be removed from the N and/or C terminal in this way. Fragments may also be generated by one or more internal deletions. For example, a variant of the invention may consist of or comprise two or more epitope regions from a full length polypeptide of the region in the absence of non-epitope amino acids. Preferably a fragment of a TLR9 or TLR7 polypeptide comprises at least one epitope capable of inducing an immune response against the unmodified TLR9 or TLR7 polypeptide.

The TLR9 and/or TLR7 antagonist may be a nucleic acid molecule that encodes a TLR9 and/or TLR7 polypeptide as described herein. Such a nucleic acid molecule may be used to deliver the TLR9 and/or TLR7 polypeptide to the individual and the polypeptide may be expressed from the nucleic acid in vivo or in vitro. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

A polypeptide antagonist of TLR9 and/or TLR7 may thus be produced from or delivered in the form of a polynucleotide which encodes, and is capable of expressing, it. Polynucleotides can be synthesised according to methods well known in the art, as described by way of example in Sambrook et al (1989, Molecular Cloning—a laboratory manual; Cold Spring Harbor Press). Substantially pure antigen preparations can be obtained using standard molecular biological tools. That is, polynucleotide sequences coding for the above-described moieties can be obtained using recombinant methods, such as by screening cDNA and genomic libraries from cells expressing an antigen, or by deriving the coding sequence for a polypeptide from a vector known to include the same. Furthermore, the desired sequences can be isolated directly from cells and tissues containing the same, using standard techniques, such as phenol extraction and PCR of cDNA or genomic DNA. See, e.g., Sambrook et al., supra, for a description of techniques used to obtain and isolate DNA. Polynucleotide sequences can also be produced synthetically, rather than cloned.

The nucleic acid molecules of the present invention may be provided in the form of an expression cassette which includes control sequences operably linked to the inserted sequence, thus allowing for expression of the polypeptide of the invention in vivo in a targeted subject species. These expression cassettes, in turn, are typically provided within vectors (e.g., plasmids or recombinant viral vectors) which are suitable for use as reagents for nucleic acid immunization. Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

Thus, a polypeptide antagonist of TLR9 and/or TLR7 may be provided by delivering such a vector to a cell and allowing transcription from the vector to occur.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extrachromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters, such as β-actin promoters, may be used. Tissue-specific promoters are especially preferred. Mammalian promoters include the metallothionein promoter which can be induced in response to heavy metals such as cadmium.

In one embodiment a viral promoter is used to drive expression from the polynucleotide. Typical viral promoters for mammalian cell expression include the SV40 large T antigen promoter, adenovirus promoters, the Moloney murine leukaemia virus long terminal repeat (MMLV LTR), the mouse mammary tumor virus LTR promoter, the rous sarcoma virus (RSV) LTR promoter, the SV40 early promoter, the human cytomegalovirus (CMV) IE promoter, adenovirus, including the adenovirus major late promoter (Ad MLP), HSV promoters (such as the HSV IE promoters), or HPV promoters, particularly the HPV upstream regulatory region (URR). All these promoters are readily available in the art.

In one embodiment, the promoter is a Cytomegalovirus (CMV) promoter. A preferred promoter element is the CMV immediate early (IE) promoter devoid of intron A, but including exon 1. Thus the expression from the polynucleotide may be under the control of hCMV IE early promoter. Expression vectors using the hCMV immediate early promoter include for example, pWRG7128, and pBC12/CMV and pJW4303. A hCMV immediate early promoter sequence can be obtained using known methods. A native hCMV immediate early promoter can be isolated directly from a sample of the virus, using standard techniques. U.S. Pat. No. 5,385,839, for example, describes the cloning of a hCMV promoter region. The sequence of a hCMV immediate early promoter is available at Genbank #M60321 (hCMV Towne strain) and X17403 (hCMV Ad169 strain). A native sequence could therefore be isolated by PCR using PCR primers based on the known sequence. See e.g Sambrook et al, supra, for a description of techniques used to obtain and isolate DNA. A suitable hCMV promoter sequence could also be isolated from an existing plasmid vector. Promoter sequences can also be produced synthetically.

In some embodiments, the polynucleotide, expression cassette or vector will encode an adjuvant, or an adjuvant will otherwise be provided. As used herein, the term “adjuvant” refers to any material or composition capable of specifically or non-specifically altering, enhancing, directing, redirecting, potentiating or initiating an antigen-specific immune response. A suitable adjuvant may be an ADP-ribosylating bacterial toxin. These include diphtheria toxin (DT), pertussis toxin (PT), cholera toxin (CT), the E. coli heat labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile and Staphylococcus aureus EDIN. Most ADP-ribosylating bacterial toxins contain A and B subunits.

Polynucleotides of interest may be used in vitro or in vivo in the production of a peptide of the invention. Such polynucleotides may be used as described herein as TLR9 and/or TLR7 antagonists, such as in the prevention, treatment or diagnosis of liver cancer.

Gene therapy and nucleic acid immunization are approaches which provide for the introduction of a nucleic acid molecule encoding one or more selected antigens into a host cell for the in vivo expression of the antigen or antigens. Methods for gene delivery are known in the art. See, e.g., U.S. Pat. Nos. 5,399,346, 5,580,859 and 5,589,466. The nucleic acid molecule can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells which have been removed from a subject. In this latter case, cells containing the nucleic acid molecule of interest are re-introduced into the subject such that an immune response can be mounted against the antigen encoded by the nucleic acid molecule. The nucleic acid molecules used in such immunization are generally referred to herein as “nucleic acid vaccines.”

In one embodiment, the vector itself may be a recombinant viral vector. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors. In the case of viral vectors, administration of the polynucleotide is mediated by viral infection of a target cell.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin™, and DOTAP (1,2-bis(oleyloxy)-3-(trimethylammonio)propane), see, e.g., Felgner et al. (1987) Proc. Natl. Acad. Sci. USA 84:7413-7416; Malone et al. (1989) Proc. Natl. Acad. Sci. USA 86:6077-6081; U.S. Pat. Nos. 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publication No. WO 93/19768).

Alternatively, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The formulated compositions will include an amount of the molecule (e.g. vector) of interest which is sufficient to mount an immunological response. An appropriate effective amount can be readily determined by one of skill in the art. Such an amount will fall in a relatively broad range that can be determined through routine trials. The compositions may contain from about 0.1% to about 99.9% of the vector and can be administered directly to the subject or, alternatively, delivered ex vivo, to cells derived from the subject, using methods known to those skilled in the art.

Where the TLR9 and/or TLR7 antagonist is a polypeptide, nucleic acid or vector as described herein that is intended to provoke the immune system of the individual to responds to TLR9 and/or TLR7, the antagonist may be administered in an amount sufficient to elicit an immune response to one or more epitopes of a polypeptide of the invention and/or to alleviate, reduce, cure or at least partially arrest symptoms and/or complications from a disease, such as liver cancer, which is associated with TLR9 and/or TLR7 expression.

Prophylaxis or therapy can be accomplished by a single direct administration at a single time point or by multiple administrations, optionally at multiple time points. Administration can also be delivered to a single or to multiple sites. Those skilled in the art can adjust the dosage and concentration to suit the particular route of delivery. In one embodiment, a single dose is administered on a single occasion. In an alternative embodiment, a number of doses are administered to a subject on the same occasion but, for example, at different sites. In a further embodiment, multiple doses are administered on multiple occasions. Such multiple doses may be administered in batches, i.e. with multiple administrations at different sites on the same occasion, or may be administered individually, with one administration on each of multiple occasions (optionally at multiple sites). Any combination of such administration regimes may be used.

In one embodiment, the same or different compositions of the invention may be administered at different sites or on different occasions as part of the same treatment regime. In such methods, multiple administrations of the same composition may be given. Multiple different compositions may be administered. For example, two or more different antagonists as described herein may be administered in two or more different compositions. In one embodiment, the two or more antagonists comprise at least one TLR7 antagonist and at least one TLR9 antagonist. The TLR7 and TLR9 antagonist may be the same antagonist or different antagonists. It is known that improved immune responses may be generated to an antigen by varying the vectors used to deliver the antigen. There is evidence that in some instances antibody and/or cellular immune responses may be improved by using the same or different vectors administered sequentially as a “prime” and a “boost”.

In such a prime-boost protocol, one or more administrations of the prime and/or the boost may be performed. For example, the prime and/or boost step may be achieved using a single administration or using two or more administrations at different sites and/or on different occasions. In one embodiment, two administrations on different occasions are given for the prime step and a single administration on a later occasion is given for the boost step. The schedule and timing of such multiple administrations can be optimised for a particular composition or compositions by one of skill in the art by routine trials.

### Screening Methods

The present invention also provides methods for the identification of agents suitable for use in the treatment of liver disease. For example, the invention provides methods for the identification of antagonists of TLR9 and/or TLR7 which are suitable for use in treating or preventing liver cancer, such as hepatocellular carcinoma or cholangiocarcinoma. Antagonists identified by this method may be antagonists of TLR9 and/or TLR7 having any of the characteristics or effects described above. Antagonists identified by the methods described herein may be suitable for use in the treatment or prevention of liver cancer or in the treatment or prevention of any of the conditions or symptoms described herein.

Accordingly, the invention provides a method of identifying an agent for use in the treatment or prevention of liver cancer, the method comprising determining whether a test agent is capable of decreasing the activity or expression of TLR9 and/or TLR7. For example, the method may involve determining whether a test agent is capable of decreasing the amount or activity of TLR9 and/or TLR7, wherein the ability to decrease the amount or activity of TLR9 and/or TLR7 indicates that the compound may be suitable for use in treating or preventing liver cancer as described herein.

The method may comprise assessing the amount or activity of TLR9 and/or TLR7 in a particular cell or tissue type. This may be any cell of tissue that expresses TLR9 and/or TLR7. For example, the method may assess the amount or activity of TLR9 and/or TLR7 in the liver or in tissue or cells derived from the liver; in the kidney or heart or cells derived from the kidney or heart; in inflammatory cells, platelets or neurons; or in any other cell or tissue that expresses TLR9 and/or TLR7. The method may assess the amount or activity of TLR9 and/or TLR7 in a liver cancer tissue such as a sample of tissue from a liver cancer or a hepatocellular carcinoma or cholangiocarcinoma.

A test agent for use in a screening method of the invention refers to any compound, molecule or agent that may potentially antagonise TLR9 and/or TLR7. The test agent may be, or may comprise, for example, a peptide, polypeptide, protein, antibody, polynucleotide, small molecule or other compound that may be designed through rational drug design starting from known antagonists of TLR9 and/or TLR7.

The test agent may be any agent having one or more characteristics of an antagonist of TLR9 and/or TLR7 as described above.

The test agent to be screened could be derived or synthesised from chemical compositions or man-made compounds. Candidate agents may be obtained from a wide variety of sources including libraries of synthetic or natural compounds. Suitable test agents which can be tested in the above assays include compounds derived from combinatorial libraries, small molecule libraries and natural product libraries, such as display (e.g. phage display) libraries. Multiple test agents may be screened using a method of the invention in order to identify one or more agents having a suitable effect on TLR9 and/or TLR7, such as inhibition of TLR9 and/or TLR7 activity or expression.

The screening methods of the invention may be carried out in vivo, ex vivo or in vitro. In particular, the step of contacting a test agent with TLR9 and/or TLR7 or with a cell or tissue that comprises TLR9 and/or TLR7 may be carried out in vivo, ex vivo or in vitro. The screening methods of the invention may be carried out in a cell-based or a cell-free system. For example, the screening method of the invention may comprise a step of contacting a cell or tissue comprising TLR9 and/or TLR7 with a test agent and determining whether the presence of the test agent leads to a decrease in the amount or activity of TLR9 and/or TLR7 in the cell or tissue.

For example, the ability of a test agent to decrease the activity or expression of TLR9 and/or TLR7 may be tested in a host cell or tissue that expresses TLR9 and/or TLR7. For example, the amount or activity of TLR9 and/or TLR7 may be assessed in vitro, in vivo or ex vivo in the liver or in tissue or cells derived from the liver, in tissue or cells from another organ that expresses TLR9 and/or TLR7, such as the kidney or heart, or in other cells that express TLR9 and/or TLR7 such as inflammatory cells, platelets or neurons. The amount or activity of TLR9 and/or TLR7 may be assessed in vivo, ex vivo or in vitro in a liver cancer, liver tumor, hepatocellular carcinoma or cholangiocarcinoma or in cells or tissue derived from such a liver cancer, liver tumor, hepatocellular carcinoma or cholangiocarcinoma.

In such a cell-based assay, the TLR9 and/or TLR7 and/or the test agent may be endogenous to the host cell or tissue, may be introduced into a host cell or tissue, may be introduced into the host cell or tissue by causing or allowing the expression of an expression construct or vector or may be introduced into the host cell or tissue by stimulating or activating expression from an endogenous gene in the cell.

In such a cell-based method, the amount of TLR9 and/or TLR7 may be assessed in the presence or absence of a test agent in order to determine whether the agent is altering the amount of TLR9 and/or TLR7 in the cell or tissue, such as through regulation of TLR9 and/or TLR7 expression in the cell or tissue or through destabilisation of TLR9 and/or TLR7 protein within the cell or tissue. The presence of a lower TLR9 and/or TLR7 activity or a decreased amount of TLR9 and/or TLR7 within the cell or tissue in the presence of the test agent indicates that the test agent may be a suitable antagonist of TLR9 and/or TLR7 for use in accordance with the present invention in the treatment or prevention of liver cancer.

In one embodiment, such a cell based assay may be carried out in vitro or ex vivo on cells or tissue deriving from the patient to be treated. It may therefore be determined whether or not the test agent is capable of decreasing the activity or amount of TLR9 and/or TLR7 in the cells or tissue of that subject. For example, such a method may be carried out on a sample of cells or tissue from the liver of the patient.

A method of the invention may use a cell-free assay. For example, the TLR9 and/or TLR7 may be present in a cell-free environment. A suitable cell-free assay may be carried out in a cell extract. For example, the contacting steps of the methods of the invention may be carried out in extracts obtained from cells that may express, produce or otherwise contain TLR9 and/or TLR7 and/or a test agent. A cell-free system comprising TLR9 and/or TLR7 may be incubated with the other components of the methods of the invention such a test agent.

In such a cell-free method, the amount of TLR9 and/or TLR7 may be assessed in the presence or absence of a test agent in order to determine whether the agent is altering the amount of TLR9 and/or TLR7 in the cell or tissue, such as through destabilisation of TLR9 and/or TLR7 protein. In either case, the presence of a lower TLR9 and/or TLR7 activity or a decreased amount of TLR9 and/or TLR7 in the presence of the test agent indicates that the test agent may be a suitable antagonist of TLR9 and/or TLR7 for use in accordance with the present invention in the treatment of an individual having liver disease.

The contacting step(s) of the method of the invention may comprise incubation of the various components. Such incubations may be performed at any suitable temperature, typically between 4° C. and 40° C. Incubation periods may be selected for optimum activity, but may also be optimized to facilitate rapid high-throughput screening. Following the contact and optional incubation steps, the subject methods may further include a washing step to remove unbound components, where such a washing step is generally employed when required to remove label that would give rise to a background signal during detection, such as radioactive or fluorescently labelled non-specifically bound components.

Incubation in cell or cell-free assay systems may be performed in a microtiter plate (e.g. a 96-well plate or other microwell plate). Further, incubation may be performed in an automated fashion (e.g. for high-throughput screening).

A screening method of the invention may be carried out in vivo. For example, a screening method may be carried out in an animal model. In such an in vivo model, the effects of a test agent may be assessed in the liver, or in any other organs, cells or tissues that express TLR9 and/or TLR7. The animal model may be an animal model of liver cancer such as an animal model of hepatocellular carcinoma or cholangiocarcinoma. Preferably, the animal is a non-human animal such as a rat.

For example, a screening method may be carried out in an animal model of liver cancer as described in the Examples. As shown in the examples, treatment of a test animal, such as a rat, with DEN (Diethylnitrosamine) and NMOR (Nitrosomorpholine) leads to the development of HCC in the animal. In the animals described in the Examples, the tumour grade ranged from well differentiated to poorly differentiated HCC and all the animals developed fibrosis grade more than 3 (3-5). Animals treated with DEN and NMOR to stimulate HCC may therefore be used as model animals in the screening methods described herein. As also shown in the Examples, additional treatment with led to the development of well differentiated HCC. Animals receiving this DEN plus NMOR plus norfloxacin treatment showed minimal fibrosis grade from (0-2) and did not exceed 2 Animals treated with DEN and NMOR and norfloxacin may therefore also be used as model animals in the screening methods described herein. An animal model for use in according to the present invention may utilise any non-human animal, such as a rodent, such as a rat or mouse. Accordingly, the screening method of the present invention may comprise the step of administering a test agent to a model animal as described herein and determining whether the presence of the test agent leads to a decrease in the amount or activity of TLR9 and/or TLR7 in the liver or other organs, cells or tissues of the animal, and particularly in the liver cancer tissue of the animal as discussed above.

Such a model may be used to assess the in vivo effects of a test agent. For example, such a model may be used to assess whether the test agent is capable of decreasing the activity or amount of TLR9 and/or TLR7 in vivo. In such a method, the amount of TLR9 and/or TLR7 may be assessed and/or the activity of TLR9 and/or TLR7 may be assessed.

An in vivo model may also be used to determine whether the test agent has any unwanted side effects. For example, a method of the invention may compare the effects of a test agent on TLR9 and/or TLR7 with its effects on other receptors in order to determine whether the test agent is specific.

In an in vivo model as described herein, or an in vitro model such as a cell-based or cell-free assay model as described herein, the effects of a test agent on TLR9 and/or TLR7 may be compared with the effects of the same agent on other TLRs. As discussed above, a preferred TLR9 and/or TLR7 antagonist for use in a method of treatment as described herein may be an agent that antagonises TLR9 and/or TLR7, but that does not antagonise other TLRs. The screening methods of the invention may thus include an additional step of assessing whether the test agent has any effect on the activity or amount of one or more other TLRs such as one or more TLRs that are not TLR9 and/or TLR7. In such a method, a test agent may be identified as a suitable TLR9 and/or TLR7 antagonist if it is found to decrease the activity or amount of TLR9 and/or TLR7, but not to decrease, not to significantly decrease, not to significantly decrease, not to alter, or not to significantly alter, the activity or amount of one or more other TLRs in the same assay. As discussed above in relation to TLR9 and/or TLR7 antagonists, the one or more other TLRs may be selected from one or more of TLR2 and TLR4.

Where the assay is carried out in vivo, for example in an animal model as described herein, such a method may comprise comparing the amount or activity of TLR9 and/or TLR7 in the liver cancer tissue, liver or other organs of the test animal in the presence or absence of the test agent. An observation that the level or activity of TLR9 and/or TLR7 is decreased in the liver cancer tissue, liver or other organs of animals treated with the test agent suggests that the test agent may be a suitable antagonist of TLR9 and/or TLR7. A further finding that treatment with the same test agent does not significantly decrease or alter the levels or activity of one or more other TLRs receptors, such as TLR2 or TLR4, may further indicate that the test agent is a suitable specific antagonist of TLR9 and/or TLR7 that may be used in the methods of treatment described herein.

In the screening methods described herein, the presence of a lower TLR9 and/or TLR7 activity or a decreased amount of TLR9 and/or TLR7 in the presence of the test agent indicates that the test agent may be a suitable antagonist of TLR9 and/or TLR7 for use in accordance with the present invention to treat an individual having liver cancer, such as to treat or prevent liver cancer or HCC or cholangiocarcinoma.

A test agent that is an antagonist of TLR9 and/or TLR7 may result in a decrease in TLR9 and/or TLR7 activity or levels of at least 5%, at least 10%, at least 25%, at least 50%, at least 60%, at least 75%, or at least 85% or more in the presence of the test agent compared to in the absence of the test agent. A test agent that is an antagonist of TLR9 and/or TLR7 may result in a decrease in TLR9 and/or TLR7 activity or levels such that the activity or level of TLR9 and/or TLR7 is no longer detectable in the presence of the test agent. Such a decrease may be seen in the sample being tested or, for example where the method is carried out in an animal model, in particular tissue from the animal such as in the liver or in tissue from a liver tumor.

Levels or amounts of TLR9 and/or TLR7 may be measured by assessing expression of the TLR9 and/or TLR7 gene. Gene expression may be assessed by looking at mRNA production or levels or at protein production or levels. Expression products such as mRNA and proteins may be identified or quantified by methods known in the art. Such methods may utilise hybridisation to specifically identify the mRNA of interest. For example such methods may involve PCR or real-time PCR approaches. Methods to identify or quantify a protein of interest may involve the use of antibodies that bind that protein. For example, such methods may involve western blotting. Regulation of TLR9 and/or TLR7 gene expression may be compared in the presence and absence of a test agent. Thus test agents can be identified that decrease TLR9 and/or TLR7 gene expression compared to the level seen in the absence of the test agent. Such test agents may be suitable antagonists of TLR9 and/or TLR7 in accordance with the invention.

The effects of a test agent may be assessed by assessing the effects of that agent on a cell that expresses TLR9 and/or TLR7. The specificity of the agent may be assessed in a similar way, by assessing morphometry of the receptor on several cell types which express either only TLR9 and/or TLR7, or other Toll like receptors that are not TLR9 and/or TLR7, such as TLR2 and TLR4, and testing for downstream signals to determine specificity. Such experiments may be carried out using cell types that are known to express the various adrenergic receptor types. Such experiments may be carried out using cells that have been engineered to contain or express one or more Toll like receptor types, such as TLR9 that would not naturally be expressed by such cells.

### Pharmaceutical Formulations

A suitable TLR9 and/or TLR7 antagonist as described herein is typically formulated for administration with a pharmaceutically acceptable carrier or diluent. The antagonist may be any antagonist as defined herein including any antagonist identified by a screening method of the invention. The formulation may comprise one or more antagonists as described herein. The formulation may comprise one or more TLR9 antagonists as described herein. The formulation may comprise one or more TLR7 antagonists as described herein. The formulation may comprise one or more antagonists of TLR9 and TLR7 as described herein. The formulation may comprise one or more TLR9 antagonists and one or more TLR7 antagonists as described herein. The antagonist may thus be formulated as a medicament with a standard pharmaceutically acceptable carriers) and/or excipient(s) as is routine in the pharmaceutical art. The exact nature of the formulation will depend upon several factors including the desired route of administration. Typically, the antagonist may be formulated for oral, intravenous, intragastric, intravascular or intraperitoneal administration.

The pharmaceutical carrier or diluent may be, for example, an isotonic solution such as physiological saline. Solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with ornithine and at least one of phenylacetate and phenylbutyrate, a pharmaceutically acceptable carrier, e.g. sterile water, olive oil, ethyl oleate, glycols, e.g. propylene glycol, and if desired, a suitable amount of lidocaine hydrochloride.

Where the antagonist to be administered is a nucleic acid molecule, for example where the antagonist is in the form of an expression vector, certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules.

A pharmaceutical formulation in accordance with the present invention may further comprise one or more additional therapeutic agents. For example, the formulation may comprise one or more TLR9 and/or TLR7 antagonists as defined herein. The formulation may comprise one or more TLR9 and/or TLR7 antagonists as described here and also one or more additional therapeutic agents. Preferably the additional therapeutic agent(s) are agents which will assist in the treatment or prophylaxis of the individual to be treated. For example, one or more agents that are effective at treating liver cancer or other liver disease may be administered as part of a formulation as described herein. One or more agents that are effective at treating an underlying liver condition or symptom thereof in the patient may be administered as part of a formulation as described herein.

### Treatment

The present invention provides methods for the treatment of individuals having liver cancer, particularly for the treatment or prevention of symptoms and conditions associated with or resulting from HCC or cholangiocarcinoma. Accordingly, the invention provides a method of treating or preventing liver cancer comprising administering to said subject an antagonist of TLR9 and/or TLR7. Similarly, an antagonist of TLR9 and/or TLR7 may be provided for use in a method of treating or preventing liver cancer. Also provided is the use of an antagonist of TLR9 and/or TLR7 in the manufacture of a medicament for use in the treatment or prevention of liver cancer. In any embodiment described herein, the liver cancer may be HCC or cholangiocarcinoma.

The antagonist may be any antagonist as described herein including any antagonist identified by a screening method of the invention. The antagonist may be provided in a formulation as described herein. An antagonist of TLR9 and/or TLR7 as described herein is thus administered to a subject in order to treat or prevent liver cancer, or particular symptoms or conditions associated with liver cancer in the subject. An antagonist of TLR9 and/or TLR7 as described herein can thus be administered to improve the condition of a subject, for example a subject suffering from liver cancer An antagonist of TLR9 and/or TLR7 as described herein may be administered to alleviate the symptoms of a subject, for example the symptoms associated with liver cancer. An antagonist of TLR9 and/or TLR7 as described herein may be administered to combat or delay the onset of liver cancer or any symptom associated therewith. The invention can therefore prevent the medical consequences of liver cancer. The individual may be at risk of liver cancer, for example due to chronic liver disease such as cirrhosis. The individual to be treated may be identified as being at risk of liver cancer by the presence of one of more risk factors selected from viral hepatitis caused by infection with hepatitis B or hepatitis C, heavy alcohol consumption, obesity, non-alcoholic steatohepatitis, exposure to carcinogenic toxins (e.g. aflatoxins) or carcinogenic drugs, hemochromatosis, autoimmune hepatitis, α1-antitrypsin deficiency and glycogen storage disease type 1. A TLR9 and/or TLR7 antagonist as described herein may be used in the prophylactic treatment of liver cancer, i.e. to prevent the onset or progression of liver cancer, in an individual having one or more of these risk factors. The methods described herein may be used to prevent or delay the onset of liver failure in such a patient, such as a patient having cirrhosis. Use of an antagonist of TLR9 and/or TLR7 as described herein may thus extend the life of a patient with liver disease, such as a patient having, or at risk of liver cancer such as HCC or cholangiocarcinoma.

The treatment of liver cancer, or the treatment of an individual having liver cancer, as described herein, refers to the treatment of an individual having liver cancer or an individual at risk of liver cancer. The individual may be suffering from liver failure, such as acute liver failure (ALF) or acute on chronic liver failure (ACLF). The individual may be suffering from chronic liver disease such as cirrhosis. The individual may be suffering from alcoholic hepatitis or non-alcoholic steatohepatitis.

The individual may be suffering from, or at risk of, one or more symptoms or conditions caused by or associated with liver cancer. Any one or more of these conditions or symptoms may be treated or prevented in accordance with the present invention. The methods and uses described herein may be of utility in the treatment or prevention of any one or more of these symptoms or conditions in an individual suffering from liver disease.

As described herein, the antagonist of TLR9 and/or TLR7 may lead to decreased expression and/or decreased levels of TLR9 and/or TLR7 in the liver of the subject. For example, the antagonist may be an agent that inhibits transcription of TLR9 and/or TLR7 in cells of the subject.

As described herein, the antagonist of TLR9 and/or TLR7 may lead to decreased activity of TLR9 and/or TLR7 in the liver of the individual.

As described herein, the antagonist of TLR9 and/or TLR7 may stimulate or result in the generation of an immune response in the individual which acts against endogenously expressed TLR9 and/or TLR7.

The subject is treated with an antagonist of TLR9 and/or TLR7 as described herein. As described above, the antagonist of TLR9 and/or TLR7 may be administered alone or in the form of a pharmaceutical formulation or composition. The formulation or composition may comprise one or more antagonists of TLR9 and/or TLR7 and may comprise one or more additional therapeutic or prophylactic agents.

As discussed above, two or more different TLR9 and/or TLR7 antagonists as described herein may be used in combination to treat a subject. The two or more antagonists may be administered together, in a single formulation, at the same time, in two or more separate formulations, or separately or sequentially as part of a combined administration regimen.

An antagonist or formulation of the invention may be administered by any suitable route. Preferably it is administered by oral, intravenous, intragastric, intraperitoneal or intravascular routes. The antagonist or formulation may be administered directly to the liver of the subject.

The antagonist is administered in a therapeutically effective amount. A suitable dose of an antagonist of the invention can be determined according to various parameters such as the age, weight and condition of the subject to be treated; the type and severity of the liver disease; the route of administration; and the required regimen. A suitable dose can be determined for an individual antagonist. For example, for some antagonists a typical dose may be in the order of from 0.1 mg/kg/day to 30 g/kg/day. A physician will be able to determine the required dosage of antagonist and for any particular subject.

The present invention is broadly applicable to therapeutic methods and is relevant to the development of prophylactic and/or therapeutic treatments. It is to be appreciated that all references herein to treatment include curative, palliative and prophylactic treatment.

Prophylaxis or therapy includes but is not limited to eliciting an effective decrease in TLR9 and/or TLR7 amount, function or activity in order to cause a reduction in one or more symptoms or conditions associated with, or resulting from, liver cancer. The symptoms or conditions may be, for example, any of those discussed above. Prophylaxis or therapy may result in the prevention or delay of such symptoms, such as the prevention or delay of liver cancer in an individual at risk of such liver cancer, such as an individual with cirrhosis or with one or more of the risk factors discussed above. Prophylaxis or therapy may result in the maintenance of one or more symptoms or conditions associated with liver cancer in a patient where such symptoms or conditions have been increasing or are expected to increase as a result of the liver cancer. Prophylaxis or therapy may result in the maintenance of a particular level of such symptoms or conditions in a patient where such symptoms or conditions have been increasing or in which such symptoms or conditions are expected to increase as a result of the liver cancer. Prophylaxis or therapy may result in such changes in symptoms or conditions in such an individual changing at a reduced rate compared to the changes that would have been seen or would have been expected in the absence of such treatment.

Prophylaxis or therapy may have similar effects in relation to any of the symptoms or consequences of liver cancer described herein. That is, treatment in accordance with the present invention may lead to a lessening in the severity of such symptoms or consequences, maintenance of an existing level of such symptoms or consequences or a slowing or reduction in the worsening of such symptoms or consequences.

### Patients to be Treated

The present invention relates to the prevention and treatment of liver cancer such as hepatocellular carcinoma (HCC) and cholangiocarcinoma in individuals in need thereof. An individual to be treated in accordance with the present invention may therefore have liver cancer such as HCC or cholangiocarcinoma or may be at increased risk of liver cancer such as HCC or cholangiocarcinoma. For example, the subject may have cirrhosis. The subject may have portal hypertension. The subject may have one or more of the following risk factirs: viral hepatitis caused by infection with hepatitis B or hepatitis C, heavy alcohol consumption, obesity, non-alcoholic steatohepatitis, exposure to carcinogenic toxins (e.g. aflatoxins) or carcinogenic drugs, hemochromatosis, autoimmune hepatitis, α1-antitrypsin deficiency and glycogen storage disease type 1.

The individual to be treated may have been diagnosed as suffering from one or more of these risk factors, or one or more symptoms or conditions as described herein that may be associated with such risk factors, for example by any of these methods. The individual to be treated may have been diagnosed as being at risk of any of these risk factors or such symptoms or conditions. For example, the individual may have been diagnosed with one or more symptoms that are associated with cirrhosis. For example, the individual to be treated may have liver cirrhosis, alcoholic hepatitis, idiopathic non-cirrhotic portal hypertension, congenital hepatic fibrosis, partial nodular transformation, Budd-Chiari syndrome, portal vein thrombosis, right heart failure or schistosomiasis infection. The methods described herein may be used to prevent liver cancer in a patient having cirrhosis and/or any one or more of the risk factors described herein. An individual suffering from any of these risk factorss may be treated in accordance with the present invention. The present invention may be used to treat, prevent or ameliorate the effects of any of these risk factors.

The subject to be treated may be any individual which is susceptible to liver cancer such as HCC or cholangiocarcinoma. The subject may be male or female. Women may be more susceptible to the adverse effects of alcohol than men. Women can develop alcoholic chronic liver disease in a shorter time frame and from smaller amounts of alcohol than men.

The subject to be treated may be a human. The subject to be treated may be a non-human animal. The subject to be treated may be a farm animal for example, a cow or bull, sheep, pig, ox, goat or horse or may be a domestic animal such as a dog or cat. The subject may or may not be an animal model for liver disease. The animal may be any age, but will often be a mature adult subject.

### Diagnostic Methods

The present invention also relates to methods for diagnosis or assessing the prognosis of a liver cancer such as HCC or cholangiocarcinoma. In all the methods described herein, the liver cancer is preferably HCC or cholangiocarcinoma. An individual to be diagnosed may be individual as described above in relation to individuals to be treated. For example, a method of diagnosis as described herein may be carried out on an individual who is at risk of liver cancer, such as an individual having one or more of the risk factors as described herein. The individual may exhibit one or more symptoms of liver cancer. A diagnostic method as described herein may be used to assist a physician in assessing or determining the prognosis of an individual as described herein that has liver cancer or is at risk of liver cancer.

As explained in the Examples, the expression of TLR9 and TLR7 is seen in liver tumor tissue. This can be used in the identification of, or diagnosis of, liver cancer. In particular, the expression of TLR9 and TLR7 is seen in the cytoplasm in liver tumor tissue such as HCC tissue or cholangiocarcinoma tissue. No expression or only a very low level of TLR9 and TLR7 expression is seen in normal liver tissue and in liver tissue from patients having cirrhosis or hepatitis. As mentioned above, chronic hepatitis can progress to cirrhosis which can progress to HCC. This increased expression in HCC but not in hepatitis or cirrhosis can be used to diagnose HCC. Thus, the expression of TLR9 and TLR7 in a sample from the liver of an individual may indicate that the sample included tumor tissue, and that the individual is therefore suffering from liver cancer, such as HCC or cholangiocarcinoma. The invention thus provides a method of detecting, diagnosing or identifying the presence of, liver cancer such as HCC or cholangiocarcinoma, the method comprising: (a) providing a sample of liver from an individual, and (b) determining whether TLR9 and TLR7 is expressed in said sample, such as in the cytoplasm of liver cells in the sample, wherein the expression of TLR9 and TLR7 in said sample, such as in the cytoplasm of liver cells in the sample, indicates that the individual may be suffering from liver cancer. Preferably these methods are carried out in vitro on a sample such as a biopsy that has been obtained from the individual.

As demonstrated in the Examples, expression of TLR9 and TLR7 in liver cancer tissue is found particularly in the cytoplasm. In a method as described herein, the cellular location of any TLR9 and TLR7 expression may also be expressed. Thus, a method of detecting, diagnosing or identifying the presence of, liver cancer such as HCC or cholangiocarcinoma, may comprise: (a) providing a sample of liver from an individual, and (b) determining whether TLR9 and TLR7 is expressed in the cytoplasm of cells in said sample, wherein the expression of TLR9 and TLR7 in the cytoplasm of cells in said sample indicates that the individual may be suffering from liver cancer.

As demonstrated in the Examples, it has also been found that the amount of TLR9 expression seen in liver cancer tissue correlates with the degree of proliferation in that tissue. The amount of TLR9 and TLR7 expression may therefore be used as an indicator for the rate of proliferation or the rate of growth of a liver cancer. For example, the invention provides a method of determining the rate of growth of a liver cancer comprising: (a) providing a sample of liver from an individual including some tumor tissue, and (b) assessing the level of TLR9 and TLR7 expression in said sample (such as the level of cytoplasmic expression of TLR9 and TLR7 in cells of the sample), wherein the level of TLR9 and TLR7 in said sample is indicative of the rate of growth of the cancer. The level of TLR9 and TLR7 expression may be compared with known controls, such as the level of TLR9 and TLR7 expression in a sample of normal liver from the same individual or from another individual not suffering from liver cancer. The level of TLR9 and TLR7 expression may be compared with the level of TLR9 and TLR7 expression in one or more samples from known individuals wherein the rate of proliferation of the liver cancer in those individuals was known, or the level of TLR9 and TLR7 expression in known samples in which the rate of proliferation has been assessed using other means, such as by using a different proliferation marker such as Ki-67 or in which the rate of apoptosis has been assessed, for example by detecting an apoptotic marker such as caspase-3. This can be used to assess the relative rate of proliferation of the liver cancer in the individual of interest.

Expression of TLR9 in liver cancer was seen at a greater level at the proliferating edges of the tumor. This observation can be used to determine which areas of a tumor are proliferating, or whether a tumor is actively proliferating at its edges, i.e. whether the liver cancer is spreading or growing. For example, the invention provides a method of determining whether a liver cancer is spreading comprising: (a) providing a sample from a liver cancer in the individual, (b) determining the level of TLR9 and TLR7 expressed in the sample, wherein a greater level of TLR9 and TLR7 expressed in one part of the sample or in a sample obtained from one part of the tumor indicates that the cancer is likely to be spreading or growing at that part of the sample or tumor. For example, the sample may be from the edge of a liver cancer tumor and the presence of increased expression of TLR9 and TLR7 in that edge region of the tumor may indicate that the edge of the tumor is undergoing proliferation, and that the tumor is therefore growing or spreading. This may be used to gather information about the prognosis of the individual suffering from the liver cancer and this information may be used by a physician in determining a suitable treatment for that individual, for example in determining the area of liver tissue that should be surgically removed.

### EXAMPLES

### Example 1

### Immunohistochemistry

The tissue used was tissue microarray paraffin embedded sections, purchased from Vbiolabs (Cambridge, UK). Each slide contained 102 liver tissue cores. 9 cores were from normal tissue, 26 cores were from hepatitis B and C positive liver tissue, 25 cores were from cirrhosis patients and 42 cores were taken from cancerous liver tissue. Slides were stained for TLR2 (ab47840), TLR4 (76B357.1), TLR9 (ab12121), caspase3 and KI67 (ab16667) using kits purchased from Abcam, UK (except TLR4, kits purchased from Lifespan Bioscience).

1. Paraffin was removed by placing the slides in an oven for one hour prior to processing. Fresh xylene was then added (four changes, each for 5 minutes).
2. Sections were rehydrated using: One change of absolute ethyl alcohol (5 minutes); One change of 90% ethyl alcohol (5 minutes); One change of 80% ethyl alcohol (5 minutes); One change of 70% ethyl alcohol (5 minutes).
3. The slides were washed in PBS for 5 minutes.
4. For antigen retrieval: (a) A plastic container was filled with sufficient antigen retrieval solution (citrate buffer, pH6) and heated in a microwave oven until boiling. (b) The slides were then placed in the antigen retrieval solution and heated in microwave oven for 10 minutes. (c) The container was removed from the oven and allowed to cool for 20-30 minutes to reach room temperature.
5. Slides were placed in phosphate buffered saline (PBS) for 2 minutes (repeated three times).
6. Excess liquid was removed by carefully tapping the edge of each slide on paper towels. It was important to dry the slides around sections using gauze pad without drying the sections themselves.
7. Endogenous peroxidase activity was blocked for 30 min with hydrogen peroxide (10%).
8. Slides were placed in phosphate buffered saline (PBS) and washed for 2 minutes (repeated three times).
9. Excess liquid was removed by carefully tapping the edge of each slide on tissue paper.
10. Blocking agent (bottle 1 of the Invitrogen kit) was used for 30 minutes.
11. Slides were washed with PBS+Tween 20 (0.05%) for 2 minutes (repeated three times).
12. Primary antibodies were added to the slides (one antibody per slide) and the slides incubated overnight at 4° C. Antibodies were as follows: mouse TLR2 (dilution 1:500), mouse TLR4 (1/100), mouse TLR9 (1/200) and rabbit KI67 (1/100).
13. Secondary antibodies were placed on the slides and incubated for 30 minutes: HRP was used for the mouse antibody and AP for the rabbit antibody (kit from Invitrogen).
14. Slides were washed with PBS+Tween 20 (0.05%) for 2 minutes (three repeats).
15. For the rabbit antibody we use the fast red one tablet in 5 ml substrate (AP) for the Mouse antibody we use the DAB (HRP).
16. Slides were washed with tap water for 5 minutes.
17. Nuclear counter staining was performed using Dako Hematoxylin (Hx) Cat no (2020) for 3 minutes.
18. Slides were washed with tap water for 20 minutes.
19. Slides were mounted with Aqueous mounting media (Dako) and kept in the oven for 30 minutes.

The scoring system was carried out by two pathologists. For cytoplasmic staining (TLR2, TLR7, TLR9 and VEGF) we evaluated the intensity of the staining and the extent on a scale from 0-3. For staining intensity: 0: negative, 1: weak staining, 2: moderate staining intensity and 3: high staining intensity. For the staining extent, 1: ≦⅓ cells stained, 2; >⅓<⅔ the cells are stained and 3; ≧⅔ of the cells are stained. For the membranous staining a scale of 0 to 3 was used where 0=no membranous staining, 1=weak membranous staining, 2=moderate membranous staining and 3=high intensity membranous staining. For nuclear staining we gave a score 0 for absence and 1 for the presence.

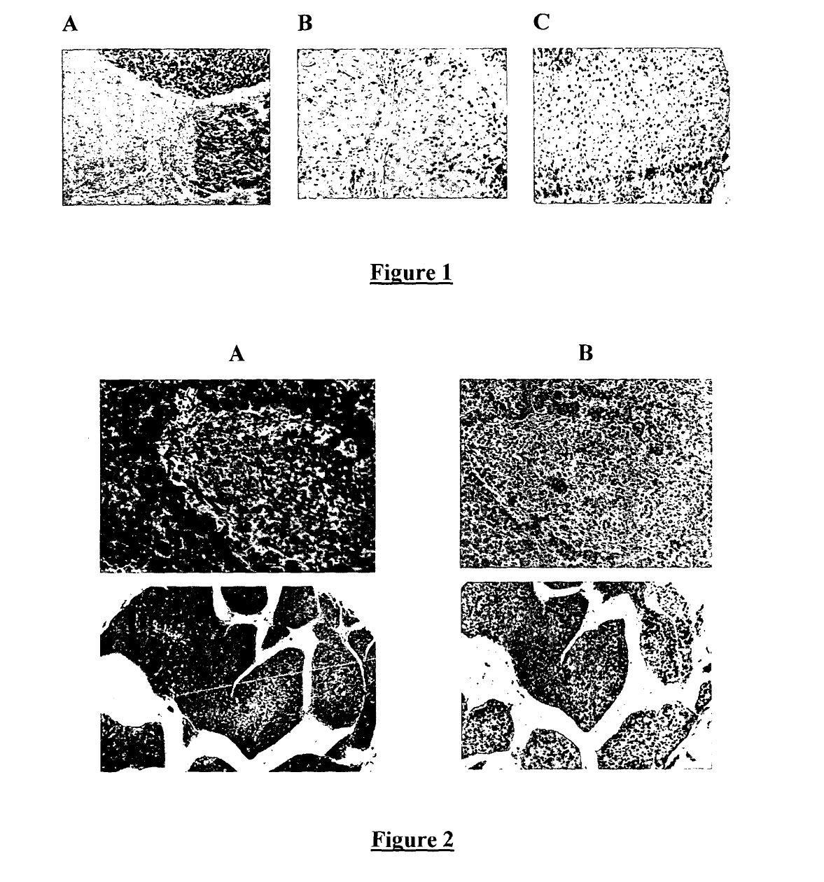

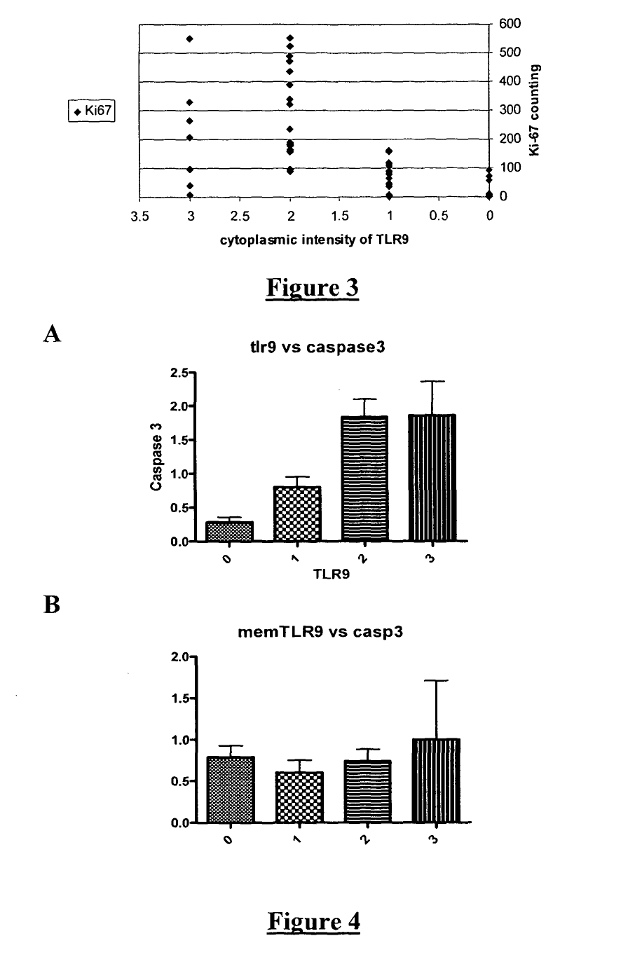

No significant difference in staining for TLR2 or TLR4 was seen between normal and HCC liver samples. In contrast, staining for TLR9 was significantly higher in HCC than in normal liver. Membranous expression of TLR9 was found in only 2 cases out of 9 in normal liver and 12 out of 26 cases of hepatitis. It was noted that it was in the cytoplasm with very faint expression (+1) in 4/26 cases of hepatitis (which may be due to viral infection as those cases are hepatitis C or B). In cirrhosis it was found in the hepatocytes membranes in 13/25 cases. On the other hand it was found at grade (+1) in 17/42 cases of the HCC, at grades (+2) and (+3) in 17/42 cases and negative in 8 cases. Some cases of HCC showed membranous staining (13/42). It was noticed also that the membranous staining of HCC associated with the +1 cytoplasmic staining except in 4 cases, where it was associated with cytoplasmic staining +2. (See FIG. 1). The same tissue array cores were stained with Ki-67. Scoring was performed in a blind fashion by two individual pathologists. For the quantitation of Ki-67 expression, we counted the positively stained nuclei among 1000 hepatocytes in the highest expression area using a standardized grid. (See FIG. 2). As shown in FIG. 3, there was a close correlation between the proliferative index (Ki-67 staining) and TLR-9 staining; r=0.8, p<0.001. The proliferative index was <100, 100-200, >200 in TLR9 negative, weak and moderate-to-strong cases, respectively. We found also a positive correlation between TLR9 cytoplasmic staining and Caspase 3 with a p<0.0001 with one way ANOVA test (FIG. 4). However there was no significant correlation between membranous TLR9 staining and caspase 3.

TLR9 staining was seen in the membranes in inflammation and cirrhosis and it appears to become cytoplasmic with the development of HCC. Significant correlation was seen between the intensity of cytoplasmic staining for TLR9 and the presence of the proliferation marker Ki-67. Significant correlation was also seen between cytoplasmic staining for TLR9 and the presence of the apoptotic marker Caspase 3.

### Example 2

### Cells in Culture

The aim of the study was to assess the expression of TLR9 in cell lines of the following cell cultures (HEPG2, Huh 7, HUCCT). We used frozen cell lines at the beginning of the experiment. Medium=RPMI with L-glutamine (Gibco, Invitrogen) (500 ml), penicillin streptomycin 10.000 (5 ml) and FBS heated inactivated (50 ml). Thawing the cells: All the cell lines were frozen. 40 ml of the media without FBS was warmed in a 37° C. water bath half an hour before the start. The cells were also warmed until they became liquid (defrosted) and put it in the warm media. The cells were then centrifuged (10 minutes, 1500 cycle/min, 37° C.). The media was discarded. 4 ml of media was placed with FBS and the alive cells counted using a hemocytometer. After being sure of the number of good alive cells we put the cells in a flask containing 20 ml of media with FBS. Cultures were incubated at 37° C. and CO₂ 5%. Once the cells were growing, culture medium was removed and discarded the next day. Cells were washed with media without FBS. 20 ml of new media with FBS was added to the flask and incubated. When the cells become a confluent layer all the media in the flask was removed and rinsed with media without FBS. 5 ml 0.05% Trypsin—EDTA was added. Cultures were incubated at 37° C. and CO₂ 5% for 10 minutes. 5 ml of media containing FBS was added to stop the reaction of Trypsin. 400 μl the cell suspension was added to a new culture flask with 20 ml of media with FBS. Cultures were incubated at 37° C. and CO₂ 5%. Cells were cultured in flasks and the cells collected after 24 h and after 48 hours. After 24 h incubation, cells were trypsinized using 0.05% trypsin for 10 minutes. Media with FBS was added to stop the trypsin reaction. Cells were centrifuged and media added. For every cell culture cells were counted using 10⁶ cells. Antibodies for TLR2, TLR4 and TLR9 were used following the protocol below. The antibodies used were TLR2-PE-Cy7 (3 μl), TLR4-APC (20 μl) and TLR9-PE (3 μl). At the same time we used isotypes for each antibody as a positive control. Cells were taken out so every vial has a specific cell line, with the number of cells as 10⁶. TLR4 and TLR2 were added to each vial and incubated for 30 min in the dark at room temperature. 500 μl of FACS permebilisation solution was added to each sample and samples incubated at room temperature in the dark again for 10 minutes. Samples were centrifuged (5 min, 4° C., ×1800 rpm). FACS solution was discarded. TLR9 was added to each sample and incubated again for 30 minutes in the dark at room temperature. 1 ml of PBS was added to each sample and the samples centrifuged again (5 minutes, 4° C., 1800 rpm). The PBS was discarded and the results analysed.

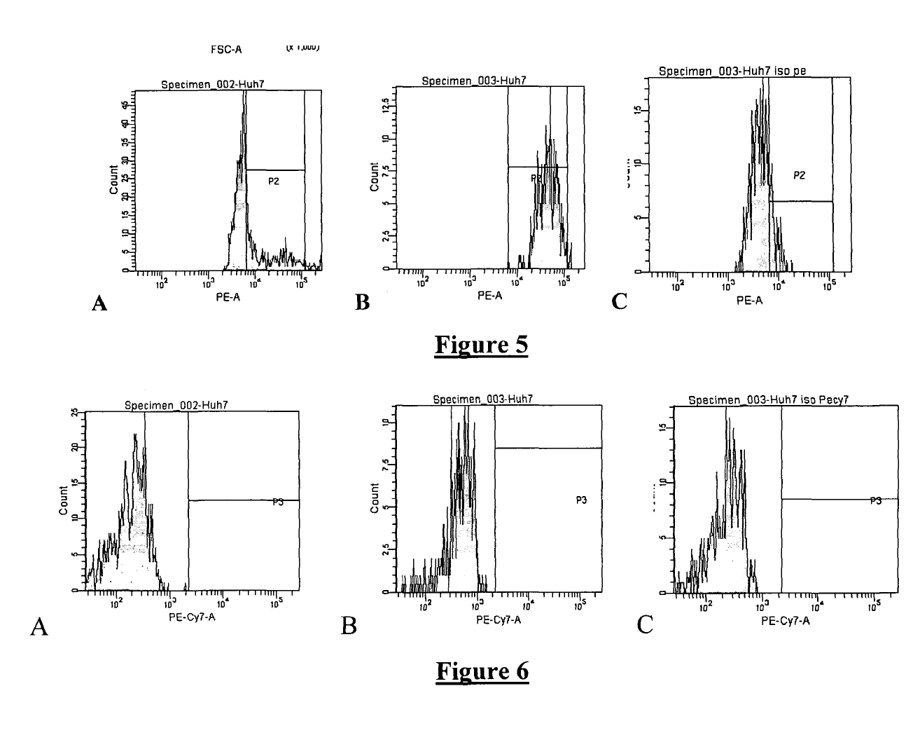

We found that after 24 h there was high TLR9 in all the cell lines specially the Huh7 (FIG. 5). After 48 hours we repeated the experiment and we found that the amount of TLR9 expression was increased compared to 24 h and also compared to the isotype. It was also high in Hu7 compared to the other cell line compared to the isotype (FIG. 6).

TLR9 expression was high in cell lines (HEPG2, Huh7 and Hucct. Intensity increased with increased with cell proliferation. Neither TLR2 nor TLR4 showed any significant increase from isotype or the stage of cell culture.

### Example 3

### Animal Model for HCC

We chose a Fisher Rat to be our model. We started with 18 Fisher rats, which were 5 weeks old on the beginning of the experiment. We divided them into 3 groups. They were acclimatised for one week before the experiment at room temperature 29-32° C. and humidity 60%-65%. The first group used 6 Fisher rats as an HCC model. On the day we started we weighed them and determined the exact dose for each rat.

We started with a single dose of DEN (Diethylnitrosamine™, sigma) using 100 mg/kg as single intraperitoneal injection. Then we put NMOR (Nitrosomorpholine™, Sigma) in the concentration of 80 ppm in the water for 14 weeks. The second group, another 6 Fisher rats, got the same HCC model treatment (DEN and NMOR) plus we gavaged them with norfloxacin in the dose of 20 mg/kg per day. The third group, at the same time, was 6 rats with a control of just plain water. We weighed them every other day and stopped treatment on the 14^{th} week, then left them for two more weeks until the 16^{th} week. We noticed the two groups who took the DEN and NMOR didn't increase in weight at the same rate as the control.

When we started our experiment the rats average weights were 212-241 g in the 1^{st} group, 225-260 g and 221-249 g in the 2^{nd} and 3^{rd} groups respectively. At the end of experiment for the groups that took the carcinogenecic treatment average weights were 328-353 g and 330-372 g respectively. For the control group the average weight was 430-478 g. DEN and NMOR group after 14 weeks treatment: liver shows multinodules and irregular surface with areas of hemorrhage. Liver shows bridging necrosis and fibrosis beside the tumour cells. DEN, NMOR and Norfloxacin group: liver shows less nodules than the first group. Liver shows minimal fibrosis beside the tumour cells. Control group: Smooth bright outer surface of the liver.

All the animals in the first group developed HCC. The tumour grade ranged from well differentiated to poorly differentiated HCC and all the animals developed fibrosis grade more than 3 (3-5). All animals in the 2^{nd} group developed well-differentiated HCC except one, which showed very early grade of HCC, which is considered high-grade dysplasia. All of them showed minimal fibrosis grade from (0-2) and did not exceed 2. In the control group, all the animals had normal livers.

### Example 4

### Increased Expression of TLR7 and 9 in Hepatocellular Carcinoma

By immunohistochemistry technique on paraffin embedded sections this technique depends on the detection of antigen by using appropriate antibody target the specific antigen. The paraffin embedded sections have been processed in xylene to remove the wax then rehydrated in differential descending alcohol. 100%, 95%, 70% and then PBS. After antigen retrieval with citric acid solution in microwave, the peroxidase is blocking with 3% hydrogen peroxide. The antibody has been added overnight in 4° C. then appropriate secondary HRP is added then the DAB for colour detection of antigen antibody complex. TLR 9 antibody: mouse monoclonal antibody (Abcam ltd.); TLR 7 antibody: rabbit polyclonal antibody (Abcam ltd.); Secondary antibody (DAKO ltd)

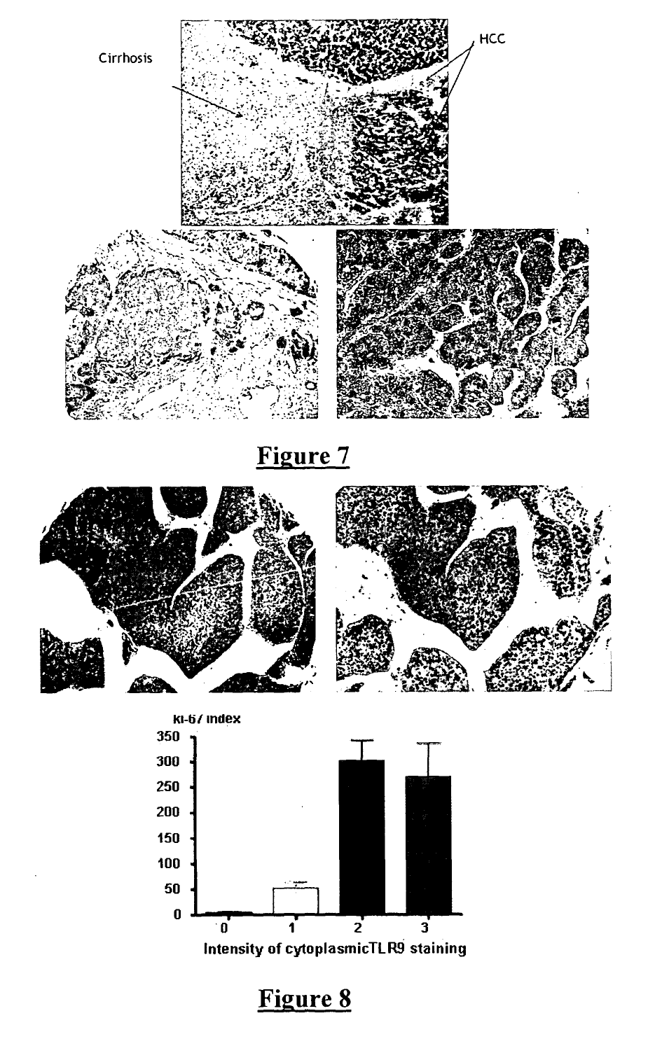

TLR9 was found in the membranes and in the cytoplasm of the hepatocytes on tissue microarray slides (FIG. 7). In normal liver: we found membranous TLR9 in only 2 cases out of 9 but no cytoplasmic staining. In hepatitis: 12/26 of hepatitis cases showed membranous staining of TLR9. Faint cytoplasmic TLR9 expression (+1) was found in 4/26 cases of hepatitis (those cases are hepatitis due to C or B). In cirrhosis: we observed membranous TLR9 expression in hepatocytes in 13/25 cases. In HCC: We found +1 staining in 17/42 cases of HCC and in 17/42 there was +2 and +3 presence. 8 cases were negative. Also, some cases (13/42 of HCC) showed membranous staining. It was noticed that the membranous staining of HCC was associated with the +1 cytoplasmic staining except in 4 cases which were associated with +2 cytoplasmic staining.

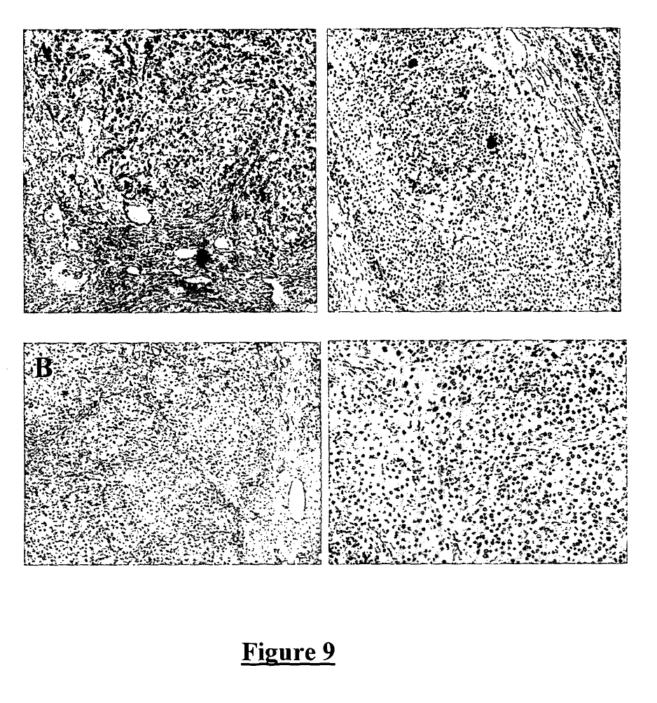

As shown in FIG. 8, there was significant correlation between the intensity of cytoplasmic TLR9 and the proliferation index which is measured by the positive ki-67 hepatocytes among 1000 hepatocytes; r=0.8, p<0.001. TLR7 was found highly expressed in the nuclei and perinuclear area in 16/20 cases of HCC (see FIG. 9). In cirrhosis some staining was found in 4/20 cases (p<0.001).

TABLE 1
TLR7 staining in liver biopsies from Hepatocellular
carcinoma and cirrhosis.

| | HCC | Cirrhosis |
|---|---|---|
| Antibody | n = 20 | n = 20 |
| TLR7 | 16 | 4 |

### Example 5

### Effect of Administering Chloroquine to a Rodent Model of HCC

40 five weeks old male fisher rats were studied. They were divided into 2 groups, Group 1 contained 15 rats who were taking DEN and NMOR alone. Group 2 comprised of 25 rats who were taking DEN, NMOR and Chloroquine was administered in addition. The rats have been given 100 mg/kg DEN (Diethylnitrosamine) (sigma) once intraperitoneal. Nitosomorpholine (NMOR) was been supplied in water with concentration of 80 ppm. Chloroquine dose is 25 mg/kg/day.

Liver and blood were collected from the animals after 8, 10 and 12 weeks respectively. In the Chloroquine treated group only 1/10 (10%) animals developed evidence of hepatocellular carcinoma but in the untreated group 8/9 (89%) animals developed Hepatocellular carcinoma (p<0.0001) suggesting that Chloroquine prevents the occurrence of Hepatocellular carcinoma in these animals. (Table 2)

TABLE 2

| Week | HCC in DEN& NMOR | HCC in Chloroquine treated |
|---|---|---|
| 8^{th} | 2/3 | 1/4 |
| 10^{th} | 3/3 | 0/3 |
| 12^{th} | 3/3 | 0/3 |

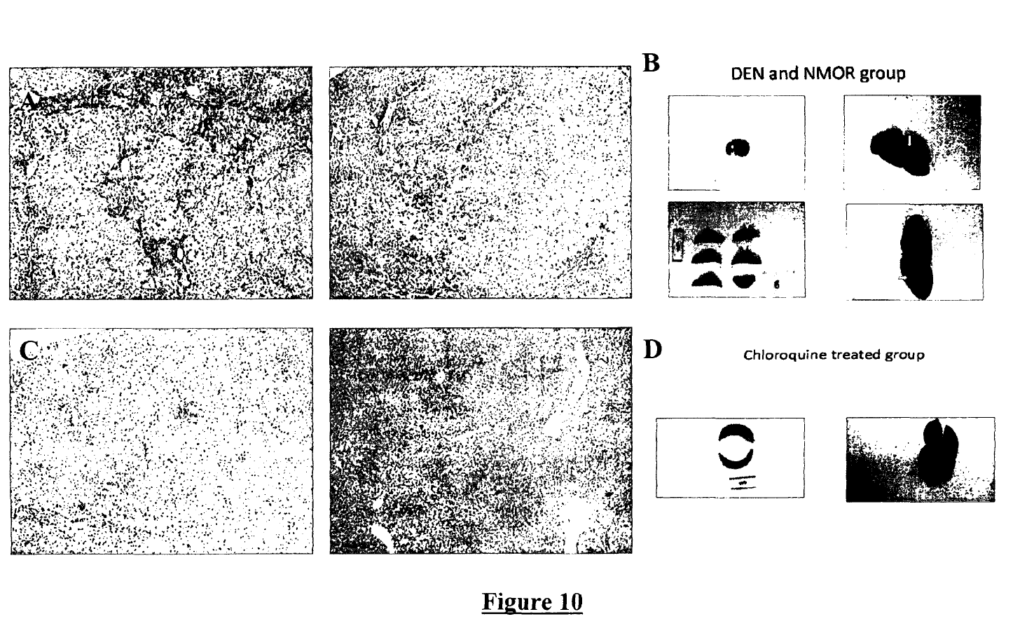

As shown in FIG. 10, in the group treated with DEN and NMOR alone without any chloroquine there was frank microscopic (A) and macroscopic appearance of hepatocellular carcinoma which was not seen in the DEN and NMOR group that were administered chloroquine either microscopically (C) or macroscopically (D)

### Example 6

### Treatment of Cholangiocarcinoma Implantation in Immunodeficient Mice (NOD-SCID) with a TLR7 and TLR9 inhibitor (IRS954, Dynavax) or Chloroquine

12 NOD-SCID mice were injected intrahepatically with 5×10⁶ cells of HUCCT (cholangiocarcinoma). 4 of them were control, 4 were given chloroquine with a dose of 25 mg/kg in water supply and 4 were given IRS 954 (Dynavax) intraperitoneally 50 μl once weekly. After 50 days a mass was found subcutaneously. There was a significant reduction of tumour size in Chloroquine (p<0.001) and a TLR7 and 9 inhibitor (IRS 954, Dynavax) (p<0.01) treated mice comparing with control.

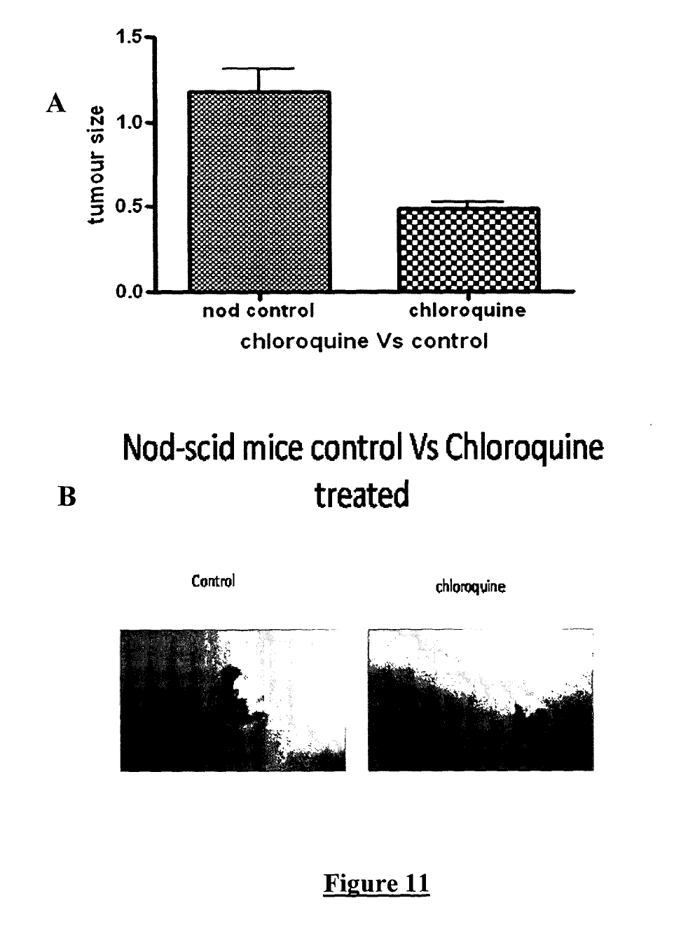

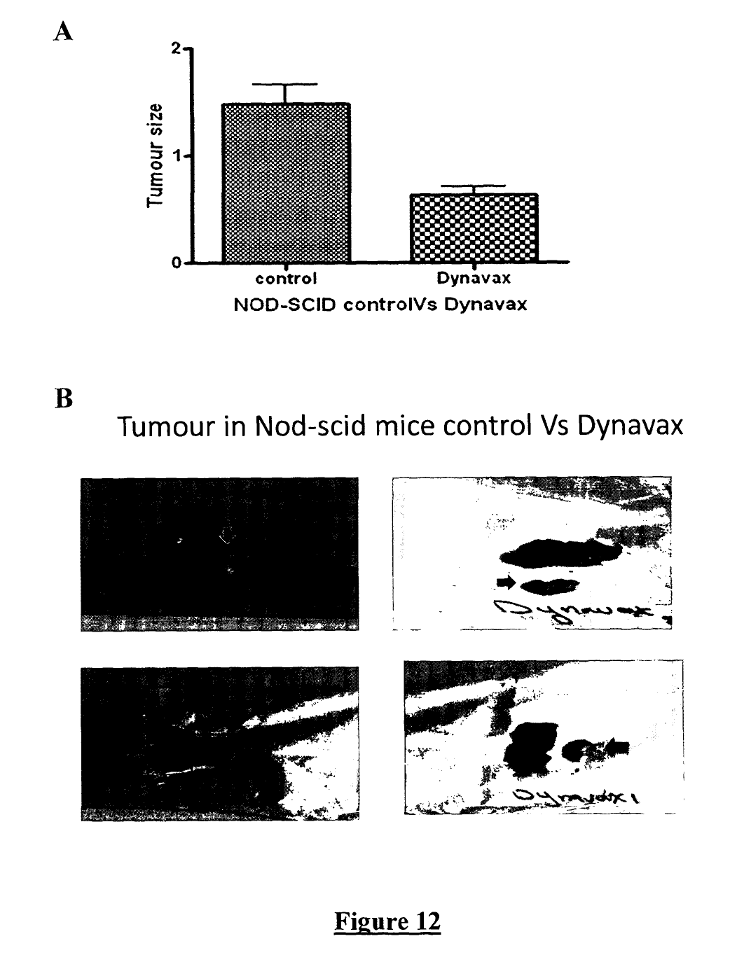

As shown in FIG. 11, the size of the tumour was significantly less in the group treated with chloroquine (A). This panel shoes the actual nodule in an animal treated with chloroquine and an untreated control (B). As shown in FIG. 12, the size of the tumour was significantly less in the group treated with the TLR7 and 9 inhibitor IRS 954 (Dynavax) (A). This panel shoes the actual nodule in an animal treated with IRS 954 and an untreated control (B).

### Example 7

### Effect of Stimulation and Inhibition of TLR 9, TLR 7 and 9, and Treatment With Chloroquine of Isolated Hepatocellular Carcinoma, Hepatoblastoma and Cholangiocarcinoma Cells in Culture

Using different techniques, manual cell counting, nucleo-counter machine and Promega proliferation assay. Using 10⁴ cellsplate them in 96 wells plate treat some of them with cpg (TLR9 stimulator; Invivogen ODN 2006, Cat No Tlr1-2006-1), iODN (TLR9 inhibitor (ttaggg)₄ (classII), Enzo life science cat no ALX-746-351), Chloroquine (Invivogen) and comparing them with the control untreated cells. In Huh7, HepG2 and HUCCT

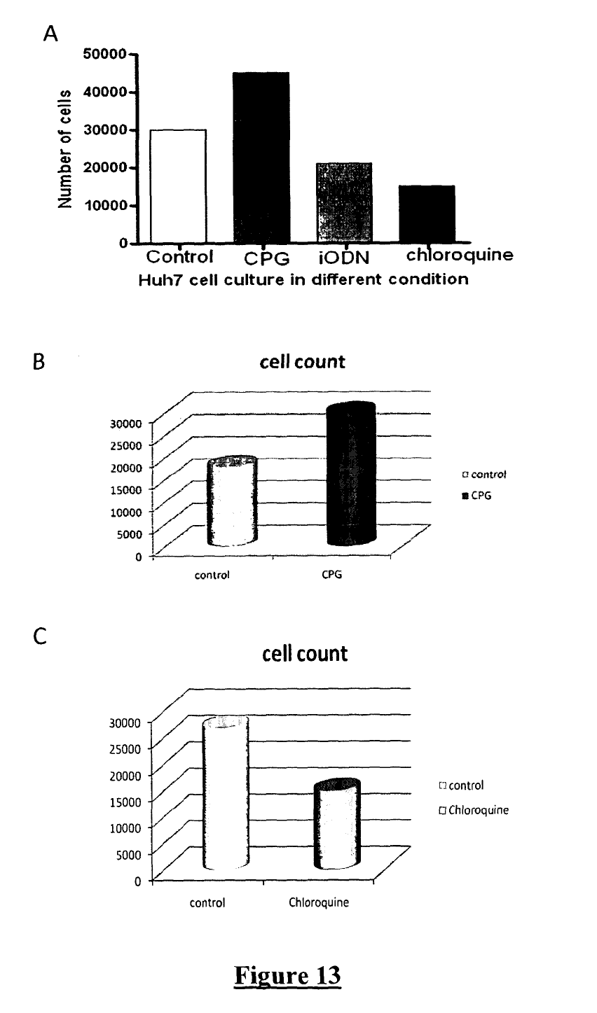

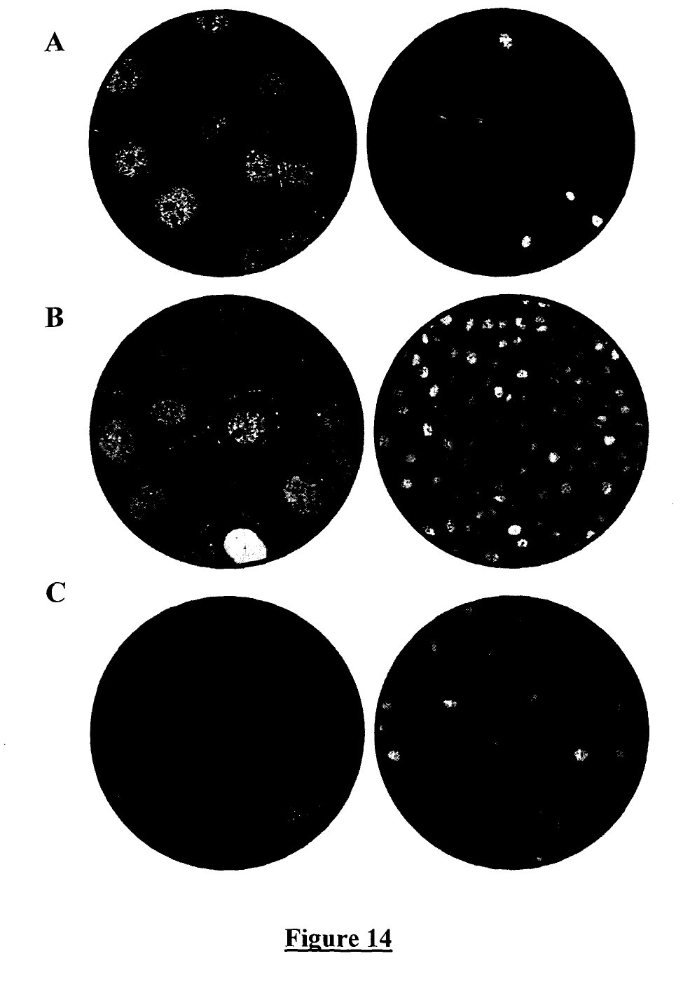

FIG. 13. (A) This panel shows that in a hepatocellular carcinoma cell line (HuH7), TLR 9 stimulator, cpg was associated with evidence of proliferation which was significantly inhibited in the presence of specific iODN (TLR9 inhibitor) and chloroquine. (B) TLR9 stimulation with cpg of HepG2 cells increased cell proliferation. (C) Treatment of HepG2 cells with Chloroquine reduced cell proliferation.

### Example 8

### Intracellular Localisation of TLR7 and TLR9 in Hepatocellular Carcinoma Cell Lines and the Effect of Stimulation with cpg or Inhibition by Inhibitory Oliginucleotide (iODN)

Confocal microscopy was performed after incubating Huh 7 cells (Hepatocellular Carcinoma cell line) with anti TLR7 and TLR9 antibody. The experiments were repeated following incubation with TLR9 stimulator cpg (as in Example 7) or inhibitory oligonucleotide (iODN, as in Example 7).

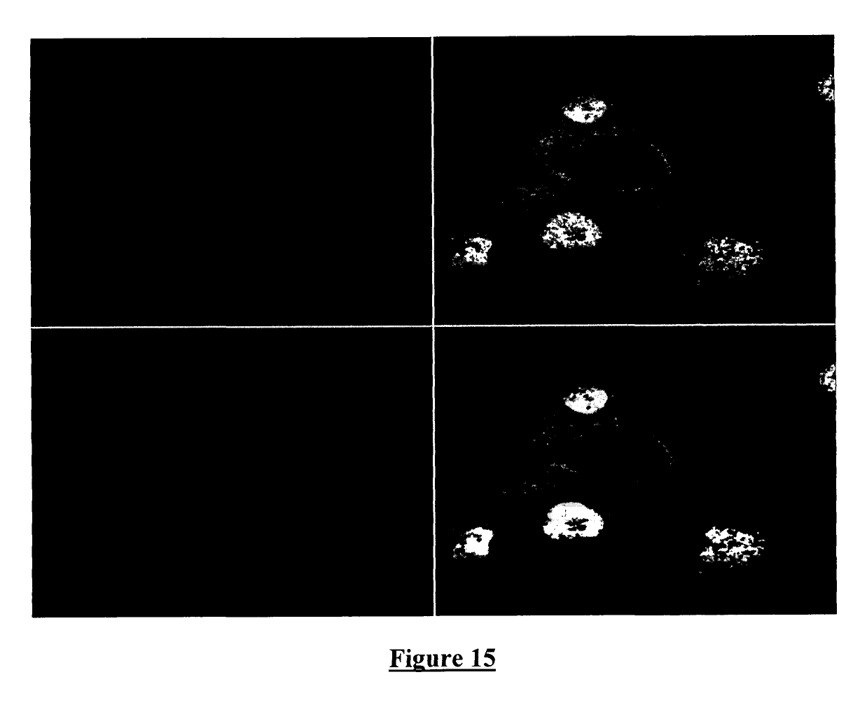

FIG. 14 shows (A) TLR9 localisation within the cell localised to the cytoplasm, plasma membrane, endosome and nucleus. (B) TLR9 localisation within the cell localised to the cytoplasm, plasma membrane, endosome and nucleus which has increased markedly following stimulation with TLR9 stimulatory cpg. This confocal image shows TLR9 localisation within the cell localised to the cytoplasm, plasma membrane, endosome and nucleus which has increased markedly reduced following administration of TLR9 inhibitory oligonucleotide (iODN). FIG. 15. This confocal image shows TLR7 localisation within the cell localised to the cytoplasm, plasma membrane, endosome and nucleus.

1. A method of treating or preventing liver cancer comprising administering to an individual in need thereof an antagonist of Toll like receptor 9 (TLR9) and/or Toll like receptor 7 (TLR7).

2. A method according to claim 1 wherein said antagonist leads to:
(a) decreased expression of TLR9 in the liver of the individual; and/or
(b) decreased levels of TLR9 in the liver of the individual; and/or
(c) decreased activity of TLR9 in the liver of the individual.

3. A method according to claim 1 wherein said antagonist leads to:
(a) decreased expression of TLR7 in the liver of the individual; and/or
(b) decreased levels of TLR7 in the liver of the individual; and/or
(c) decreased activity of TLR7 in the liver of the individual.

4. A method according to claim 1 wherein the antagonist is an antagonist of TLR9 and TLR7.

5. A method according to claim 1 wherein said antagonist is selected from chloroquine, hydroxychloroquine, quinacrine, bafilomycin A, DV1079, IMO3100, CPG52364 and IRS954.

6. A method according to claim 1 comprising administering to an individual in need thereof an agent capable of stimulating an immune response against TLR9 and/or TLR7.

7. A method according to claim 1 wherein said liver cancer is hepatocellular carcinoma (HCC) or cholangiocarcinoma.

8. A method according to claim 1, said method comprising a step of administering to said individual
(a) an antagonist of Toll like receptor 9 (TLR9), and/or
(b) an antagonist of Toll like receptor 7 (TLR7), and/or
(c) an antagonist of both TLR9 and TLR7, and/or
(d) an agent capable of stimulating an immune response against TLR9 in the individual, and/or
(e) an agent capable of stimulating an immune response against TLR7 in the individual.

9. A method of detecting liver cancer comprising
(a) providing a sample of liver from an individual
(b) determining whether TLR9 and/or TLR7 is expressed in said sample
wherein the expression of TLR9 and/or TLR7 in said sample indicates that the individual may be suffering from liver cancer.

10. A method according to claim 9 further comprising determining whether a liver cancer is spreading comprising by
(a) providing a sample from the edge of a liver cancer in the individual
(b) determining the level of TLR9 expressed in the cancer
wherein a greater level of TLR9 expressed at the edge of the cancer indicates that the cancer is more likely to be spreading.

11. A method according to claim 9 further comprising determining the rate of growth of a liver cancer comprising by
(a) providing a sample of liver from an individual including some tumor tissue
(b) assessing the level of TLR9 expression in said sample
wherein the level of TLR9 in said sample is indicative of the rate of growth of the cancer.

12. A method according to claim 9 wherein said liver cancer is hepatocellular carcinoma or cholangiocarcinoma.

13. A method of identifying an agent suitable for use in treating liver cancer, the method comprising determining whether a test agent is capable of decreasing the amount or activity of TLR9 and/or TLR7, wherein the ability to decrease the amount or activity of TLR9 and/or TLR7 indicates that the compound may be suitable for use in treating liver cancer.

14. A method according to claim 6 wherein said liver cancer is hepatocellular carcinoma (HCC) or cholangiocarcinoma.

15. A method according to claim 10 wherein said liver cancer is hepatocellular carcinoma or cholangiocarcinoma.

16. A method according to claim 11 wherein said liver cancer is hepatocellular carcinoma or cholangiocarcinoma.
